(19) United States
(12) Patent Application Publication
(10) Pub. No.: US 2024/0074775 A1
(43) Pub. Date: Mar. 7, 2024

(54) METHODS AND DEVICES FOR DELIVERING AGENTS TO THE RESPIRATORY SYSTEM

(71) Applicants: WOMEN'S AND CHILDREN'S LOCAL HEALTH NETWORK INC., North Adelaide, South Australia (AU); THE UNIVERSITY OF ADELAIDE, Adelaide, South Australia (AU)

(72) Inventors: Martin DONNELLEY, Hawthorndene, South Australia (AU); Alexandra MCCARRON, Windsor Gardens, South Australia (AU); David PARSONS, Crescent Marino, South Australia (AU)

(73) Assignees: WOMEN'S AND CHILDREN'S LOCAL HEALTH NETWORK INC., North Adelaide, South Australia (AU); THE UNIVERSITY OF ADELAIDE, Adelaide, South Australia (AU)

(21) Appl. No.: 18/261,246

(22) PCT Filed: Oct. 6, 2021
(86) PCT No.: PCT/AU2021/051164

§ 371 Date: Jul. 12, 2023

(30) Foreign Application Priority Data
Jan. 13, 2021 (AU) 2021900065

(51) Int. Cl. A61B 17/24; A61K 48/00

(52) U.S. Cl. CPC A61B 17/24; A61K 48/0075; A61B 2017/242

(57) ABSTRACT

The present disclosure relates to non-surgical methods for delivery of an agent to the respiratory system of a subject, methods of treatment using delivery of agents to the respiratory system, devices for delivery of agents to the respiratory system, and use of such devices to deliver agents to the respiratory system. In certain embodiments, the present disclosure provides a provide a non-surgical method of delivering an agent to the respiratory system of a subject. The method comprises in vivo perturbation of the airway surface in one or more parts of the respiratory system in the subject and exposing the perturbed surface of the airway to the agent, thereby delivering the agent to the respiratory system of the subject.

### PRIORITY CLAIM

This application claims priority to Australian Provisional Patent Application 2021900065 filed on 13 Jan. 2021, the contents of which are hereby incorporated by reference.

### FIELD

The present disclosure relates to non-surgical methods for delivery of an agent to the respiratory system of a subject, methods of treatment using delivery of agents to the respiratory system, devices for delivery of agents to the respiratory system and use of such devices to deliver agents to the respiratory system.

### BACKGROUND

There are a variety of pulmonary disorders that would benefit from the delivery of therapeutic agents directly to the airway or the cells of the airway. Extensive research is being undertaken in the treatment of hereditary and acquired pulmonary disorders by delivering gene therapy agents through the airway. For example, cystic fibrosis is a genetic disease resulting from two defective alleles in the CFTR gene and is an insidious disease that slowly smothers the health and potential of too many young lives. Gene therapy involving the delivery of a single copy of the CFTR gene to affected airway cells with a viral or non-viral vector continues to be explored as one of the leading ways to treat the disease.

There are also a number of other types of disorders, both pulmonary and non-pulmonary, which could benefit from the delivery of therapeutic agents to the airway. Other forms of gene therapy, such as the use of CRISPR/Cas9 based gene editing systems to correct genetic defects in airway cells are being explored. Further, other forms of therapy involving the delivery of therapeutic agents such as drugs or cells to airways may also show clinical utility.

Whilst therapeutic agents can be delivered to the respiratory system, a variety of physical, anatomical, and immune barriers have evolved to protect the airway host cells, and these natural defences affect the ability of the therapeutic agents to actually reach and act on airway cells. For example, such barriers act to limit the ability of gene vectors to transfer genes to the airway epithelium. Indeed, it appears that the major hurdles to efficient viral-vector mediated gene transfer for example, include the presence of mucus on the airway surface, a polarised epithelium, paucity of viral receptors on the apical membrane, and the presence of airway tight-junctions that prevent vectors from accessing receptors located on the basolateral side.

To address some of these issues, airway conditioning agents have been used. Whilst these agents may improve access to the airway cells, some viral vectors are very fragile due to the presence of an outer lipid envelope and can be inactivated upon contact with conditioning agents. In addition, the efficiency of viral-vector mediated delivery continues to be low.

Accordingly, alternative methods to assist with the delivery of agents to the respiratory system would be advantageous.

### SUMMARY

The present disclosure relates to non-surgical methods for delivery of an agent to the respiratory system of a subject, methods of treatment using delivery of agents to the respiratory system, devices for delivery of agents to the respiratory system, and use of such devices to deliver agents to the respiratory system.

Certain embodiments of the present disclosure provide a non-surgical method of delivering an agent to the respiratory system of a subject, the method comprising in vivo perturbation of the airway surface in one or more parts of the respiratory system in the subject and exposing the perturbed surface of the airway to the agent, thereby delivering the agent to the respiratory system of the subject.

Certain embodiments of the present disclosure provide a non-surgical method of airway delivery of an agent to a subject, the method comprising delivering the agent to the subject by exposing the agent to in vivo perturbed airway epithelium in the subject and thereby delivering the agent to the subject.

Certain embodiments of the present disclosure provide a non-surgical method of administering a therapeutic agent to a subject in need thereof, the method comprising in vivo perturbation of the airway epithelium in one or more parts of the respiratory system of the subject and exposing the perturbed airway epithelium to the agent, thereby administering the therapeutic agent to the subject.

Certain embodiments of the present disclosure provide a non-surgical method of treating a subject suffering from, or susceptible to, a disorder, the method comprising use of a device in vivo to perturb the airway epithelium in the subject and administering a therapeutic agent to the perturbed epithelium to treat the subject.

Certain embodiments of the present disclosure provide use of a device for perturbing the airway epithelium in vivo to prepare a subject for delivery of an agent to the subject.

Certain embodiments of the present disclosure provide a device for perturbing the airway surface in vivo in a subject.

Certain embodiments of the present disclosure provide a device for delivering of an agent to the respiratory system, the device comprising:
a component for perturbing a surface of the airway of the respiratory system; and a port for introducing an agent into the device to deliver the agent to the perturbed surface; and
and an outlet to permit delivery of the agent in the vehicle to the perturbed surface of the airway.

Certain embodiments of the present disclosure provide a non-surgical method of delivering an agent in vivo to a subject, the method comprising using a device as described herein to perturb the surface of the airway in the subject and exposing the perturbed surface of the airway to the agent, thereby delivering the agent to the subject.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows a wire basket used to physically perturb the tracheal airway epithelium in a rat. The basket is 1 cm in diameter at full expansion and 1.5 cm in length.

FIG. 2 shows bioluminescence images acquired 1-week following LV-FLAG-Luc-GFP delivery in PBS-sham and LPC conditioned rats. Top panel: In vivo images indicate the localisation of bioluminescence predominantly to the lung region. Middle panel: Ex vivo images of the trachea. Bottom panel: Ex vivo images of the lungs following removal of the trachea.

FIG. 3 shows in vivo and ex vivo bioluminescence flux acquired 1-week following LV-FLAG-Luc-GFP delivery in PBS-sham and LPC conditioned rats. Statistical analyses of the in vivo and ex vivo lung data revealed significant differences in flux between PBS and LPC conditioned groups, with p=0.0002 and p=0.005, respectively. Statistical analysis of the ex vivo trachea indicated there was no significant difference in flux between LPC and PBS groups, p=0.2. The plot indicates the estimated median and 95% confidence intervals. n=5-6 animals per group, Welch two sample t-test.

FIG. 4 shows ex vivo lung bioluminescence flux acquired 12 months following LV-FLAG-Luc-GFP delivery in PBS-sham and LPC conditioned rats. The dashed line indicates the limit of detection of the IVIS machine (102). No significant difference in flux was observed between animals in the PBS and LPC groups, p=0.6. n=11-12 animals per group, Wald Chi-squared test.

FIG. 5 shows comparison of enface LacZ staining using physical perturbation, chemical conditioning with 0.1% LPC or LV vector only assessed 1-week post gene transfer. Images show the trachea from each individual animal longitudinally cut into two halves to reveal the LacZ staining present within the lumen. The images display the middle portion of the trachea and are oriented to show the proximal trachea on the right. n=6 rats per group. Scale bar=1.5 mm.

FIG. 6: Physical perturbation reveals varying patterns of LacZ staining. (A, B) LacZ staining is increased in the areas overlying the inner-cartilage segments. (C-F) Basket-induced perturbation created focal regions of strong LacZ staining. High magnification images are acquired from the samples shown in FIG. 5 and indicate staining patterns present in multiple animals. Scale bars A, B=1.5 mm; C-F=0.75 mm.

FIG. 7 shows quantification of LacZ staining area from enface images shown in FIG. 5. Physical perturbation had a significantly greater area of tracheal LacZ staining after 1-week when compared to LPC conditioned (p=0.001) and LV vector only control animals (p=0.0008). There was no significant difference in the area of LacZ staining between LPC conditioned animals and those that received only LV vector (p=0.1). The plot indicates the median. n 6 animals per group, one-way ANOVA with Tukey's post-hoc test. Note that two animals are not observed on the plot as their quantified area was zero.

FIG. 8 shows histological observations in the tracheal epithelium of rats receiving physical perturbation prior to LV vector delivery assessed 1-week post gene transfer. (A) LacZ-positive surface epithelial cell types including ciliated and non-ciliated cells, and (B) transduced basal cells identified by their distinct triangular shape and contact with the basal lamina. Transduced cells within the lamina propria including suspected (C) macrophages and (D) possible fibroblasts (indicated by black arrows). While most of the tissue demonstrated full epithelial regeneration, some regions showed (E) a flattened epithelial layer lacking pseudostratification. (F) Connective tissue proliferation (fibrosis) in response to healing (denoted by asterisk). (G, H) Goblet cell hyperplasia observed at the carina. Some hyperplastic goblet cells exhibited LacZ staining. Images are shown from selected animals. Nuclear fast red counterstain. Scale bars A, C=50 μm, D-G=20 μm B, H=10 μm.

FIG. 9 shows enface images from animals receiving LPC conditioning or LV vector only indicating strong regions of LacZ staining in the proximal trachea at the site of unintentional endotracheal tube damage.

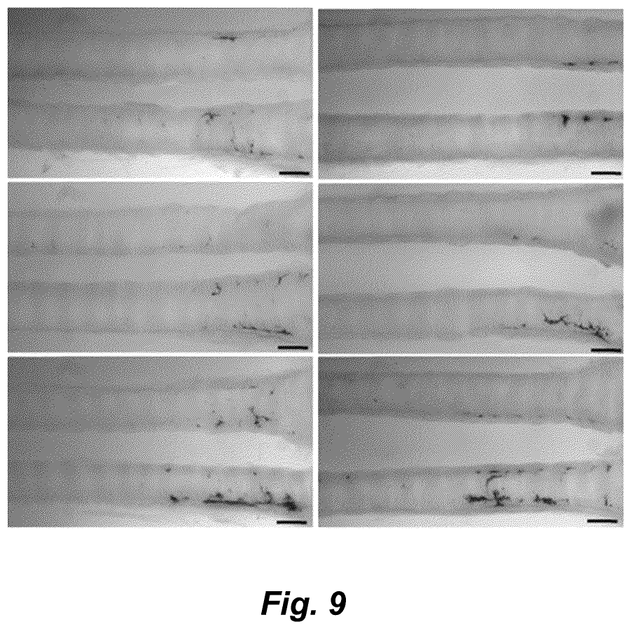

FIG. 10 shows the histology of normal rat tracheal epithelium (H&E-stained), without perturbation.

FIG. 11 shows examples of surface-cell removal by fluid jet. Low power×40.

FIG. 12 shows examples of surface-cell removal by fluid jet. High power×400.

FIG. 13 shows airway disturbance effects produced by a Cook “N-circle” wire basket.

FIG. 14 shows airway disturbance effects produced by a Cook “N-gage” wire basket.

FIG. 15 shows the effect of brush perturbation of airway surface.

FIG. 16 shows the effect of balloon tip perturbation of airway surface.

FIG. 17 shows the effect of wire-loop perturbation of rat tracheal epithelium.

FIG. 18 shows the effect of the perturbation of the airway surface via the outer surface of biopsy-forceps.

FIG. 19 shows a simultaneous LV-LacZ vector and physical airway disturbance (wired basket) stained for LacZ.

FIG. 20 shows a 40× power magnification of simultaneous LV-LacZ vector and physical airway disturbance (wired basket) stained for LacZ.

FIG. 21 shows the effect of LV-LacZ vector delivery after Cook “N-Gage” wire basket airway disturbance.

FIG. 22 shows a device for perturbing the airway surface and delivering an agent to the perturbed surface according to one embodiment of the present disclosure.

FIG. 23 shows a device for perturbing the airway surface according to another embodiment of the present disclosure.

FIG. 24 shows a bronchoscopic device for perturbing the airway surface according to a further embodiment of the present disclosure.

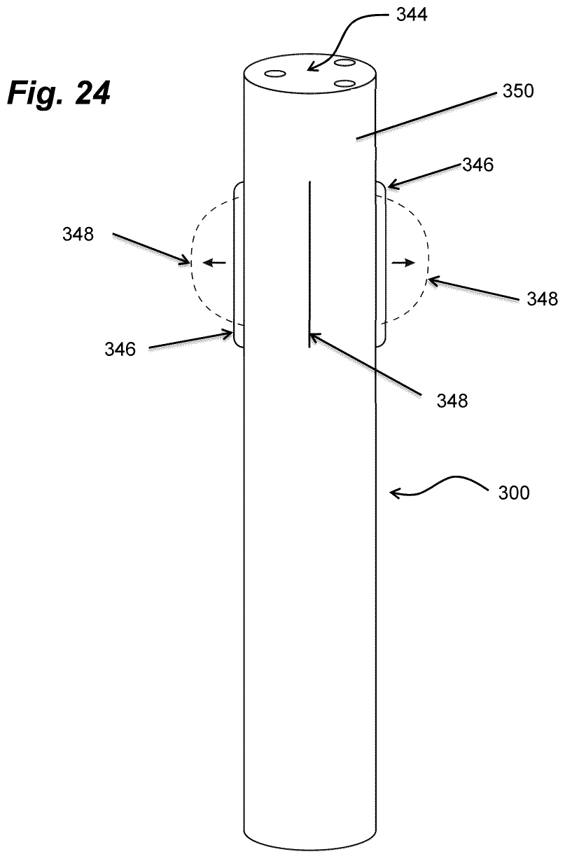

### DETAILED DESCRIPTION

The present disclosure relates to non-surgical methods for delivery of an agent to the respiratory system of a subject, methods of treatment using delivery of agents to the respiratory system, devices for preparing parts of the respiratory system for delivery of an agent, devices for delivery of agents to the respiratory system, and use of such devices to deliver agents to the respiratory system.

One or more embodiments of the present disclosure are directed to methods and devices that have one or more combinations of the following advantages: new and/or improved methods for delivering agents to the respiratory system; new and improved methods for administering agents to a subject that do not rely on traditional administration routes; non-surgical methods for delivering agents to the respiratory system; new and/or improved methods for administering therapeutic agents to the respiratory system, and methods for treatment based thereon; new and/or improved devices to prepare a subject for delivery of an agent to the respiratory system; new and/or improved methods for improving gene delivery to the respiratory system; new and/or improved methods for increasing the extent and/or duration of expression of genes delivered to the respiratory system; methods applicable for the delivery of various classes of agents to the respiratory system; methods for the delivery of existing agents and emerging agents such as mRNAs and nanoparticles by the airway; methods of delivery of agents to the respiratory system that may reduce the need for repeated re-administration of the agents, due to their improved effectiveness; methods of delivery of agents to the respiratory system that may reduce undesirable immune responses produced by repeated administration of agents to the respiratory system; methods of delivery of agents to the respiratory system that avoid the need for repeated re-administration of the agents; methods of delivery to the respiratory system that may be used to reduce the dose of an agent delivered to the respiratory system; methods of delivery to the respiratory system that may be used to reduce the manufacturing burden associated with higher doses of an agent to the respiratory system that are required using existing methods; new methods for delivering non-viral agents to a subject by airway delivery; devices for perturbing a surface of the airway to prepare for delivery of an agent to the respiratory system; devices for both perturbing a surface of the airway and delivering agents to the perturbed surface; to address one or more problems, and/or to provide one or more advantages, or to provide a commercial alternative. Other advantages of certain embodiments of the present disclosure are also disclosed herein

The present disclosure is based on the determination that in an animal model, physical perturbation of the airway significantly improved airway-based lentiviral vector mediated transduction, as compared to chemical conditioning using the lipid lysophosphatidylcholine. Physical perturbation to the airway prior to gene transfer also resulted in a 1000-fold increase in staining with a gene marker when compared to animals that did not receive any airway perturbation.

Certain embodiments of the present disclosure provide a method of delivering an agent to the respiratory system of a subject by perturbing the airway in vivo.

In certain embodiments, the present disclosure provide a non-surgical method of delivering an agent to the respiratory system of a subject by perturbing the airway in vivo.

In certain embodiments, the present disclosure provides a non-surgical method of delivering an agent to the respiratory system of a subject, the method comprising in vivo perturbation of the airway surface in one or more parts of the respiratory system in the subject and exposing the perturbed surface of the airway to the agent, thereby delivering the agent to the respiratory system of the subject.

The term “non-surgical” as used herein refers to an action on the body that does not involve cutting into the body, but rather involves an action on a part of the body that is caused by introduction into the body of a device, reagent, fluid, gas, or particulates, that results in perturbing of the airway in vivo.

In certain embodiments, the subject is a human or an animal subject. In certain embodiments, the subject is need of treatment with an agent as described herein. Delivery of agents to humans and animals is contemplated. Veterinary applications of the present disclosure are contemplated.

In certain embodiments, the respiratory system comprises one or more of the lungs, the bronchi, the bronchioles, the trachea, the pharynx, the nasal system, and the sinuses. The delivery of the agent to one or more specific parts of the respiratory system may be selected as desired, on the basis of the nature of the agent being delivered, the part(s) of the respiratory system selected for perturbation, and the desired outcome. In certain embodiments, the perturbation of the respiratory system comprises perturbation of the trachea, conducting airways and/or the lungs.

The term “perturbation” as used herein in the context of the present disclosure is to be understood to refer to a treatment or action that physically conditions an airway surface so as to make the surface more permissive to the uptake of an agent. As such, the term “perturbation” refers to a physically disruptive conditioning of an airway surface.

In certain embodiments, the method comprises perturbing a surface of the airway in the respiratory system, and delivering the agent to the subject by exposing the agent to the perturbed airway surface.

In certain embodiments, the perturbation of a surface of the airway comprises one or more of mechanical perturbation, expansive perturbation, distensive perturbation, gaseous perturbation, fluidic perturbation, chemical perturbation, enzymatic perturbation, laser light perturbation, heat perturbation and/or bronchial thermoplasty. Other types of perturbation action are contemplated.

In certain embodiments, the perturbation of a surface of the airway comprises a mechanical perturbation. In certain embodiments, the perturbation of a surface of the airway comprises an expansive perturbation. In certain embodiments, the perturbation of a surface of the airway comprises a distensive perturbation. In certain embodiments, the perturbation of a surface of the airway comprises a gaseous perturbation. In certain embodiments, the perturbation of a surface of the airway comprises a fluidic perturbation. In certain embodiments, the perturbation of a surface of the airway comprises a chemical perturbation. In certain embodiments, the perturbation of a surface of the airway comprises an enzymatic perturbation. In certain embodiments, the perturbation of a surface of the airway comprises a laser light perturbation. In certain embodiments, the perturbation of a surface of the airway comprises a heat perturbation. In certain embodiments, the perturbation of a surface of the airway comprises perturbation using a fluidic jet or a gaseous jet. In certain embodiments, the perturbation of a surface of the airway comprises a bronchial thermoplasty. In certain embodiments, the perturbation pf a surface of the airway comprises a combination of some of the aforementioned perturbations. Other types of perturbation are contemplated.

In certain embodiments, the perturbation of a surface of the airway comprises one or more of abrasive perturbation, a scraping perturbation, a furrowing perturbation, expansive perturbation, distensive perturbation, and ultrasonic perturbation, or any combination thereof. Methods for performing the aforementioned perturbation actions are known in the art and detailed herein.

In certain embodiments, the perturbation of a surface of the airway comprises delivery of a gas or fluid to the respiratory system under pressure (positive pressure or negative pressure). Methods for performing a perturbative action to the airway by delivery of gas or fluid under positive pressure are known in the art.

In certain embodiments, the perturbation of a surface of the airway comprises application of negative pressure to the airway, such as the use of suction. Methods for application of negative pressure to the respiratory system are known in the art, and include for example methods of suction.

In certain embodiments, the perturbation of a surface of the airway comprises bronchoalveolar lavage. Methods for performing bronchoalveolar lavage are known in the art.

Methods for assessing the perturbation of the airway are known in the art, and include, for example, methods such as endoscopy, real time confocal microscopy, intravital microscopy, optical coherence tomography, radial endobronchial ultrasound, methods that measure epithelial basement membrane surface area, distension parameters, leakage of liquid from the airway, and the presence of inflammatory markers (see for example Waters et al. (2012) Compr. Physiol. 2(1):1-29).

Examples of mechanical perturbation include scraping, scratching, rubbing, expansion, distension, adhesive action, abrasive action, or impactive action.

Examples of expansive or distensive perturbation include use of an expandable cage, use of an inflatable balloon, use of an expandable stent, or introduction of gas or fluid into the airway to expand or distend the airway.

Examples of gaseous perturbation include introduction of a gas under pressure into the airway, such as perturbation using a gaseous jet. The gas may be one gas, a mixture of gases (eg air), an inert gas (eg nitrogen, argon), or a gas such as oxygen or carbon dioxide.

Examples of fluidic perturbation include introduction of a fluid into the airway. Typically, fluidic perturbation will use aqueous based fluids, but non-aqueous fluids may also be utilised. For example, perturbation using a fluidic jet may be used.

Examples of chemical perturbation include introduction of a reagent into the airway to perturb the structure or function of the airway. Examples of such reagents include solvents, chelating agents, chaotropes, and detergents (denaturing, non-ionic or zwitterionic).

Examples of enzymatic perturbation include introduction of an enzyme into the airway to alter or degrade one or more protective layers, fluid or mucous layers, or cell structures present in the airway. Examples of enzymes include DNAses. Other types of enzymes are contemplated. It will be appreciated that the use of one or more enzymes to perturb the airway may involve timing or mode of delivery that is compatible with the agent to be delivered.

Examples of laser light perturbation include use of endoscope applied laser light to the airway. Appropriate emission wavelengths and power outputs may be selected.

Examples of heat or cold perturbation include endoscopic application of heat or cold to the airway, or bronchial thermoplasty.

In certain embodiments, the perturbation of the airway surface comprises perturbing one or more of airway cells, airway mucous layer, and airway surface liquid. Methods for assessing the aforementioned features are known in the art.

In certain embodiments, the perturbation of the surface of the airway comprises perturbing one or more of epithelial cells, ciliated epithelial cells, goblet epithelial cells, basal epithelial cells, ionocytes, brush cells and intermediate cells. Methods for assessing the characteristics of airway cells are known in the art.

In certain embodiments, the perturbation of the airway surface comprises disrupting the integrity of the tight junctions between the surface epithelial cells of the airway and/or partial or substantially complete removal of the surface epithelium. Methods for assessing the integrity of tight junctions are known in the art. Methods for assessing the extent of the disruption of the surface epithelium are known in the art.

Methods for exposing the airway surface to an agent include lavage, atomisation, nebulisation, aerosolization, direct instillation, bronchoscopic administration, fluidic administration, or administration by fluidic jet or gaseous jet; all of which are known in the art.

In certain embodiments, the agent may be delivered by way of a fluid, a spray, an aerosol, an atomiser or a powder. Other delivery forms are contemplated.

In certain embodiments, the agent is delivered in a vehicle. For example, the agent may be delivered in a liquid vehicle (eg saline) or in a gas (eg air).

The agent may be delivered passively to the respiratory system, or delivered to the respiratory system under pressure. Other forms for delivery are contemplated.

For example, a surface of the airway may be perturbed as described herein, and the agent delivered by an aerosol delivered by a facial mask, or delivered by way of an inhaler.

In certain embodiments, the agent is delivered by a device as described herein.

The agent may be delivered prior to perturbation of the airway surface, during perturbation of the airway surface, and/or after perturbation of the airway surface, or any combination of the aforementioned.

In certain embodiments, the agent may be delivered to the perturbed surface by way of passing through a suitable tube or a cannula, for example a tube or cannula made from a plastic (eg silicone) or a metal material, and in some embodiments utilising a Luer lock connection.

In certain embodiments, the perturbation of the airway surface comprises use of a device to apply a perturbative action directly to the airway surface. Examples of perturbative action are described herein.

In certain embodiments, the device in use produces an uncontrolled or controlled abrasive, expansive and/or distensive perturbation to the surface of the airway in the subject.

In certain embodiments, the device is used to perturb the surface of one or more of the trachea, the bronchi, and the bronchioles.

In certain embodiments, the device is a tracheobronchial device. In certain embodiments, the device is a tracheal and/or a bronchial device. In certain embodiments, the device is a bronchiolar device.

In certain embodiments, the device comprise a semi-flexible tube to permit the device to be positioned to a part of the airway to be perturbed. In certain embodiments, the device comprises a cannula system.

In certain embodiments, the device may be advanced by use of a bronchoscope (or similar).

In certain embodiments, the device comprises a bronchoscope. The use of a bronchoscope in the device permits visual inspection of the airway and selection of a suitable part of the airway for perturbation and/or delivery of the agent.

In certain embodiments, the device comprises a means for perturbing a surface of the airway. In certain embodiments, the device comprises a means for introducing the agent to the airway. In certain embodiments, the device comprises a means for perturbing a surface of the airway and a means for introducing the agent to the airway.

In certain embodiments, the device comprises an abrasive and/or an expandable component for perturbing the surface of the airway.

In certain embodiments, the device comprises a component that is a loop, a three dimensional (3D) cage, a brush, a suction device, a balloon, or a sponge. The aforementioned components may be expandible for use in the respiratory system.

For example, the device may comprise a component that is configured to be insertable into the respiratory system and expandible when deployed at the appropriate section of the airway to perturb the airway. In one embodiment, the component may be inserted into the airway when retracted into the device, and when the device has been inserted to the appropriate part of the respiratory system, the component for perturbation deployed.

In certain embodiments, the device comprises a cage, a loop or a brush configured for use in the respiratory system. In certain embodiments, the brush and/or cage are expandable, so as to permit expansion to a size suitable for perturbing a surface of the airway. The components may be kept in a compressed configuration and then allowed to expand at a suitable part of the airway to provide an abrasive and/or distensive perturbation.

In certain embodiments, the device comprises a loop for perturbing the surface of the airway. For example, the loop may be positioned to a suitable part of the airway in a retracted or compressed form, and once a suitable part of the airway has been selected, the loop expanded so that it can be used to cause abrasive and/or distensive perturbation of the airway. The loop for example may be produced from a suitable plastic or metallic material.

In certain embodiments, the device comprises a loop for perturbing the surface of the airway. For example, the loop may be positioned to a suitable part of the airway in a retracted or compressed form, and once a suitable part of the airway has been selected, the loop expanded so that it can be used to cause abrasive and/or distensive perturbation of the airway.

In certain embodiments, the device comprises a cage for perturbing the surface of the airway. For example, the cage may be positioned to a suitable part of the airway in a retracted or compressed form, and once a suitable part of the airway has been selected, the cage expanded so that it can be used to cause abrasive and/or distensive perturbation of the airway.

In certain embodiments, the device comprises a scraper for perturbing the surface of the airway. For example, the scraper may be positioned to a suitable part of the airway, and once a suitable part of the airway has been selected, the scraper deployed so that it can be used to cause abrasive perturbation of the airway.

In certain embodiments, the device comprises a brush for perturbing the surface of the airway. For example, the brush may be positioned to a suitable part of the airway in a retracted form or in a form wherein the bristles are not deployed, and once a suitable part of the airway has been selected, the brush extended or the bristles deployed so that it can be used to cause abrasive perturbation of the airway.

In certain embodiments, the device comprises an inflatable balloon configured for use in the respiratory system. The balloon may be inflated at the appropriate part of the air to provide a distensive perturbation. For example, the deflated balloon may be positioned to a suitable part of the airway, and once a suitable part of the airway has been selected, the balloon inflated so that it can be used to cause expansive perturbation of the airway.

In certain embodiments, the device comprises a sponge for use in the respiratory system. The sponge may be kept in a compressed configuration and then allowed to expand at a suitable part of the airway to provide an abrasive and/or distensive perturbation. For example, the sponge may be held in a compressed form, and once a suitable part of the airway has been selected, the sponge allowed to expand so that it can be used to cause an abrasive and/or expansive perturbation of the airway. In another example, the sponge may contain the agent and be released directly to the perturbed surface, thereby allowing the performing of simultaneous perturbation and agent delivery.

In certain embodiments, the device comprises a means for advancing and/or retracting the component for perturbing the surface of the airway. For example, the device may comprise a flexible tube having at its end a means for perturbing the airway surface, and the tube advanced or retracted within the airway by physical manipulation.

In certain embodiments, the device comprises a port for introduction of the agent so that it can be delivered to the perturbed surface of the airway. For example, the port may be used to introduce the agent in a vehicle (eg a spray or a liquid) containing the agent into the device, and the agent makes it way to the desired site of exposure.

In certain embodiments, the device comprises an outlet to permit delivery of the agent in the vehicle to the perturbed surface of the airway.

In certain embodiments, the device comprises a port to permit introduction of the agent in a vehicle into the device, and an outlet to permit delivery of the agent in the vehicle to the perturbed surface of the airway.

In certain embodiments, the airway surface is perturbed and the agent is subsequently or simultaneously exposed to the perturbed surface.

In certain embodiments, the agent is exposed to the airway surface, and then the surface of the airway is subsequently perturbed.

Examples of agents are as described herein. In certain embodiments, the agent comprises a nucleic acid, a virus, a packaged recombinant virus, a viral vector, a non-viral vector, a nanoparticle, a gene-editing agent, a small molecule, a drug, a protein, a lipid, or a cell. Other types of agents are contemplated.

In certain embodiments, the agent for delivery to the airway is a therapeutic agent.

In certain embodiments, the agent is used for gene therapy purposes. Examples include viruses or viral vectors carrying a corrective gene, recombinant nucleic acid constructs for performing gene editing (eg CRISPR/Cas9), recombinant nucleic acid constructs for expressing specific proteins of RNAs in target cells, and exosomes.

In certain embodiments, the agent is a nucleic acid. In certain embodiments, the nucleic acid comprises naked RNA or naked DNA.

In certain embodiments, the nucleic acid may be in the form of a liposome, or a nanoparticle. In certain embodiments, the nucleic acid is an encapsulated form thereof, such as lipid encapsulated RNA or DNA. Methods for preparing and using nucleic acids for delivery are known in the art.

In certain embodiments, the agent comprises a virus or a viral vector.

In certain embodiments, the virus or viral vector for delivery is selected from a lentivirus, an adenovirus, an adeno-associated virus, a helper-dependent adenovirus, a herpes virus, a retrovirus, an alphavirus, a flavivirus, a rhabdovirus or a measles virus. Other types of viruses and viral vectors are known in the art. The viruses and viral vectors may be in an encapsulated viral form, packaged in vivo or in vitro, or in a naked form. Examples of viruses and viral vectors are described in Lundstrom (2018) Diseases 6(2): 42.

In certain embodiments, the agent comprises a drug or a small molecule. In certain embodiments, the agent comprises an antibiotic, or an antifungal. Other types of drugs are contemplated.

In certain embodiments, the agent comprises a protein or an antibody (or an antigen binding part thereof).

In certain embodiments, the agent comprises a cell for delivery to the respiratory system. In certain embodiments, the cell comprises a stem cell or a progenitor cell. In certain embodiments, the stem cell is a mesenchymal stem cell or an airway basal cell. Methods for isolating and preparing cells for delivery for therapeutic purposes are known in the art.

In certain embodiments, the agent comprises exosomes. Methods for preparing exosomes are known in the art.

In certain embodiments, the subject is suffering from, or susceptible to, a disorder that would benefit from the delivery of a therapeutic agent to the respiratory system.

In certain embodiments, the subject is suffering from, or susceptible to, a genetic disease.

In certain embodiments, the subject is suffering from a pulmonary disorder that would benefit from the delivery of a therapeutic agent to the respiratory system. Examples include pulmonary or respiratory cancers such as lung cancer, chronic lung diseases, and genetic pulmonary diseases.

In certain embodiments, the subject is suffering from a non-pulmonary disorder that would benefit from the delivery of a therapeutic agent to the respiratory system. Examples include non-pulmonary cancers and immune diseases.

Examples of agents for delivery include al antitrypsin (hAAT) and Factor VIII.

In certain embodiments, the subject is suffering from, or susceptible to, a pulmonary disorder. Examples include allergic asthma, cystic fibrosis, chronic obstructive pulmonary disease, emphysema, and/or cancer. Other disorders are contemplated.

In certain embodiments, the pulmonary disease is a genetic pulmonary disorder, such as cystic fibrosis. Other disorders are contemplated.

In certain embodiments, the agent comprises a nucleic acid for expression and the method produces detectable expression from the nucleic acid in the respiratory system for 7 days or more, for 14 days or more, for 1 month, or for 6 months or more.

In certain embodiments, the method is used to deliver an agent to a subject, for administration of an agent to a subject, for gene or cell delivery to the subject, to edit a gene in a subject, to deliver therapeutic cells to the subject, or to treat a subject suffering from, or susceptible to, a disorder.

Certain embodiments of the present disclosure provide a non-surgical method of airway delivery of an agent to a subject by delivering the agent to the subject by exposing the agent to in vivo perturbed airway epithelium in the subject, as described herein.

In certain embodiments, the present disclosure provides a non-surgical method of airway delivery of an agent to a subject, the method comprising delivering the agent to the subject by exposing the agent to in vivo perturbed airway epithelium in the subject and thereby delivering the agent to the subject.

In certain embodiments, the present disclosure provides a non-surgical method of administering a therapeutic agent to a subject in need thereof by in vivo perturbing the airway epithelium in one or more parts of the respiratory system of the subject and exposing the perturbed airway epithelium to the agent, as described herein.

In certain embodiments, the present disclosure provides a non-surgical method of administering a therapeutic agent to a subject in need thereof, the method comprising in vivo perturbing the airway epithelium in one or more parts of the respiratory system of the subject and exposing the perturbed airway epithelium to the agent, thereby administering the therapeutic agent to the subject.

In certain embodiments, the present disclosure provides a non-surgical method of airway delivery of an agent to a subject, the method comprising:
perturbing a surface of the airway in the respiratory system; and delivering the agent to the subject by exposing the agent to the perturbed airway surface,
thereby delivering the agent to the subject.

Certain embodiments of the present disclosure provide a non-surgical method of treating a subject by exposing a therapeutic agent to perturbed epithelium, as described herein.

Certain embodiments of the present disclosure provide a non-surgical method of treating a subject suffering from, or susceptible to, a disorder, by use of a device in vivo to perturb the airway epithelium in the subject and administering a therapeutic agent to the perturbed epithelium to treat the subject, as described herein.

In certain embodiments, the present disclosure provides a non-surgical method of treating a subject suffering from, or susceptible to, a disorder, the method comprising use of a device in vivo to perturb the airway epithelium in the subject and administering a therapeutic agent to the perturbed epithelium to treat the subject.

Certain embodiments of the present disclosure provide use of a device for perturbing the airway epithelium in vivo to prepare a subject for delivery of an agent to the subject.

Certain embodiments of the present disclosure provide a device for perturbing the airway surface in vivo in a subject.

In certain embodiments, the device is a tracheal device. In certain embodiments, the device is a bronchial device. In certain embodiments, the device is a bronchiolar device.

Examples of devices are as described herein.

In certain embodiments, the device in use produces an uncontrolled or controlled abrasive, expansive and/or distensive perturbation to the surface of the airway in the subject.

In certain embodiments, the device comprises a bronchoscope.

In certain embodiments, the device comprises an abrasive and/or an expandable component for perturbing the surface of the airway.

In certain embodiments, the device comprises a brush and/or a cage, a loop or a brush configured for use in the respiratory system. In certain embodiments, the loop, brush or cage are expandable.

Typically, the devices will be configured for introduction of the devices into the respiratory system and then used to perturb the airway surface. For example, the device may be inserted into the respiratory system and then the device activated so as to be able to perturb the airway surface. The device will typically permit adjustment of the extent that the surface is perturbed, for example, adjustment of what degree of pressure is applied to the airway surface, and/or the area of the surface that is subject to perturbation.

Certain embodiments of the present disclosure provide a device for delivering of an agent to the respiratory system, the device comprising:
a component for perturbing a surface of the airway of the respiratory system; and
a port for introducing an agent into the device to deliver the agent to the perturbed surface.

The device typically has an outlet to permit delivery of the agent in the vehicle to the perturbed surface of the airway.

In certain embodiments, the component for perturbing a surface of the airway is retractable into the device for insertion of the device into the airway. Examples are as described herein.

Certain embodiments of the present disclosure provide a device for delivering of an agent to the respiratory system, the device comprising:
a component for perturbing a surface of the airway of the respiratory system; and
a port for introducing an agent into the device to deliver the agent to a perturbed surface; and
and an outlet to permit delivery of the agent in the vehicle to the perturbed surface of the airway.

Certain embodiments of the present disclosure provide a non-surgical method of delivering an agent in vivo to a subject, the method comprising using a device as described herein to perturb the surface of the airway in the subject and exposing the perturbed surface of the airway to the agent, thereby delivering the agent to the subject.

Standard techniques and equipment may be used for cell biology, recombinant DNA technology, molecular biology and enzymatic reactions. The foregoing techniques and procedures may be generally performed according to methods known in the art and/or as commercially available, and are as described for example in Green and Sambrook: Molecular Cloning: A Laboratory Manual (4th ed., Cold Spring Harbor Laboratory Press, Cold Spring Harbor, N.Y. (2012)) and Ausubel et al Current Protocols in Molecular Biology (2003) John Wiley & Sons, both of which are herein incorporated by reference.

The present disclosure is further described by the following examples. It is to be understood that the following description is for the purpose of describing particular embodiments only and is not intended to be limiting with respect to the above description.

### Example 1—Breaching the Delivery Barrier: Chemical and Physical Airway Epithelium Disruption Strategies for Enhancing Lentiviral-Mediated Gene Therapies

Abstract

The lungs have evolved complex physical, biological and immunological defences to prevent foreign material from entering the airway epithelial cells. These mechanisms can also affect viral and non-viral gene transfer agents, and significantly diminish the effectiveness of airway gene-addition therapies. One strategy to overcome the physical barrier properties of the airway is to transiently disturb the integrity of the airway epithelium prior to delivery of the gene transfer vector. In this study, chemical (lysophosphatidylcholine, LPC) and physical epithelium disruption approaches were assessed for their ability to improve airway-based lentiviral (LV) vector mediated transduction and reporter gene expression in rats. When assessed at 1-week post LV delivery, LPC airway conditioning significantly enhanced gene expression levels in rat lungs, while a long-term assessment in a separate cohort of rats at 12 months revealed that LPC conditioning did not improve longevity of expression. In rats receiving physical perturbation to the trachea prior to gene delivery, significantly higher gene transduction levels were found when compared to LPC-conditioned or LV-only control rats when evaluated 1-week post gene transfer. This study shows that airway epithelial disruption strategies based on physical perturbation demonstrate significant benefit for improving LV-mediated airway gene transfer in the trachea.

Introduction

Airway gene-addition therapy is currently under development for treatment of a range of hereditary and acquired pulmonary diseases. Of particular interest is the development of a gene therapy to treat airway disease caused by the common genetic disorder cystic fibrosis. Gene-addition therapy employs a vector (non-viral or viral) to deliver wild-type gene copies to the relevant airway cells, with the ultimate goal of correcting the disease pathophysiology that results from the defective gene. While vectors can be readily delivered into the lung airways, physical, anatomical, and immune barriers have evolved to protect the airway host cells against airborne pathogens. These natural defences are also directed towards gene vectors, thus limiting gene transfer to the airway epithelium.

Major hurdles to efficient viral-vector mediated gene transfer include a polarised epithelium, paucity of viral receptors on the apical membrane, and the presence of airway tight-junctions that prevent vectors from accessing receptors located on the basolateral side. One potential way to overcome these barriers is to perform epithelial perturbation prior to, or in conjunction with, gene vector delivery. Perturbation acts to disrupt tight-junction integrity, allowing vector particles access to basolateral receptors, thus enhancing vector-mediated gene transfer. Epithelial perturbation can also potentially expose cells for transduction that are not in direct contact with the airway lumen, particularly the basal stem cells.

To achieve basolateral access, airway conditioning with a ‘tight-junction opener’ can be employed to increase paracellular permeability. Airway conditioning with compounds including ethylene glycol tetraacetic acid (EGTA), perfluorochemical, sodium caprate, and sulphur dioxide inhalation have proven effective for disrupting tight-junctions and increasing viral-vector mediated gene transfer in proof-of-principle animal studies. Another compound that has been investigated extensively for this purpose is lysophosphatidylcholine (LPC). LPC is a natural component of lung surfactant that can be used to transiently open cellular tight-junctions when applied to the airway surface. A two-step dosing regimen employing LPC conditioning prior to the delivery of a HIV-1-derived lentiviral (LV) vector pseudotyped with the vesicular stomatitis virus glycoprotein (VSV-G) has previously proven effective at increasing airway gene transfer in mice.

Chemical conditioning approaches have disadvantages when it comes to performing LV-mediated gene transfer. LV vectors are highly fragile due to the presence of an outer lipid envelope and can be inactivated upon contact with conditioning agents such as LPC. Although two separate administrations—one to deliver the conditioning compound, and then a second to deliver the vector after the chemical effect occurs—can be used to enable LV vectors to take advantage of this delivery enhancement effect, this two-step process increases procedure time and complexity. Additionally, transduction may be variable as there is no guarantee that the conditioning compound and vector will localise to the same regions.

An alternative strategy to disrupt the integrity of the airway epithelium is via physical perturbation.

Early airway gene therapy studies (Pickles et al. (1996) Human Gene Therapy 7: 921-931) employing adenoviruses demonstrated that injury to the airway epithelium via use of fine forceps may be used to produce gene expression in the trachea of mice. However, these studies involved surgical methods involved gene transduction performed on tracheostomized mice, with the airway severely damaged by applying external compression of intercartilaginous regions with forceps applied externally. The studies involved anaesthesia, open neck surgery, followed by 30 mins exposure to the (Ad) vector, and tracheal surgery to enable access via two tracheostomies.

In this study we provide evidence of the effectiveness of LPC conditioning over both short-term (1-week) and long-term (12-month) durations for the first time in rat airways. The efficacy of physical airway epithelium perturbation was explored as an alternative strategy to improve in vivo gene transfer levels and this method was compared to standard LPC-conditioning and a perturbation-free LV-only control group.

Methods

Multiple treatment groups were employed in this project. The first study assessed the efficacy of LPC conditioning in rat airways to determine whether gene expression levels could be improved over short- and long-term durations. In this study, rats received LPC conditioning or a PBS (phosphate buffer saline) sham treatment, followed by a HIV-1 derived EF1α-3XFLAG-fLuc-F2A-eGFP vector (LV-FLAG-Luc-GFP) vector. In one cohort (n=6 per group) luciferase gene expression was measured 1-week following gene delivery, and in a separate cohort (n=12 per group) expression was measured 12-months after LV delivery.

To investigate and compare airway epithelial disruption strategies rats received either no epithelial disruption (control group), physical perturbation, or standard LPC conditioning, followed by delivery of a HIV-1-MPSV-nlsLacZ (LV-LacZ) vector (n=6 per group). Gene expression levels were assessed 1-week post gene transfer in this cohort of animals, as it was expected that the damaged airway epithelium would be regenerated and that gene expression would be readily apparent by this stage.

Lentiviral Vector Production

LV vector was produced using previously described methods (McCarron, A., et al. (2019) Hum Gene Ther Methods 30(3): p. 93-101; Rout-Pitt, N., et al. (2018) J Biol Methods 5(2): p. e90). The bicistronic LV-FLAG-Luc-GFP vector expressed firefly luciferase and enhanced green fluorescent protein (eGFP) genes under control of the EF1α promoter, together with three epitope FLAG tags. The LV-LacZ vector construct expressed a nuclear-localised β-galactosidase gene directed by the MPSV promoter. LV-FLAG-Luc-GFP vector was titered by quantifying the proportion of GFP positive cells using flow cytometry (McCarron, A., et al., (2019) Hum Gene Ther Methods, 2019. 30(3): p. 93-101). The titre of the LV-LacZ vector was determined using real-time quantitative PCR as previously described (Cmielewski, P., et al. (2017) Exp Lung Res, 2017. 43(9-10): p. 426-433.)

Animals

Female and male Sprague Dawley rats >8 weeks old were used. For all LV-dosing procedures, rats were anaesthetised with medetomidine (0.4 mg/kg) and ketamine (60 mg/kg) by intraperitoneal (IP) injection. Following anaesthesia, the rats were non-surgically intubated with a 16-gauge intravenous cannula (Terumo, SR-FF1651), which was positioned with the tip just past the vocal cords so that the majority of the tissue was exposed to the conditioning or perturbation and LV-vector. After procedure completion anaesthesia was reversed with atipamezole (1 mg/kg). Rats that received physical airway perturbation were given subcutaneous administration of meloxicam (1 mg/kg) for precautionary pain relief.

Intratracheal Administration of LPC and LV-Vector

Animals were placed in a supine position and received either 25 μL of 0.1% LPC (Sigma-Aldrich) or PBS. Fluid was administered to the trachea via the endotracheal (ET) tube using a gel-loading pipette tip that was lengthened with fine polyethylene tubing to reach the end of the ET tube. LV vector delivery was performed one hour following LPC conditioning (or PBS-sham) as this is the time when LPC epithelial tight-junction opening effects are apparent. LV-FLAG-Luc-GFP (1×10⁹ TU/mL) or LV-LacZ (2×10⁹ TU/mL) vectors were delivered to the trachea in two aliquots of 25 μL (50 μL total per animal).

Physical Epithelial Perturbation

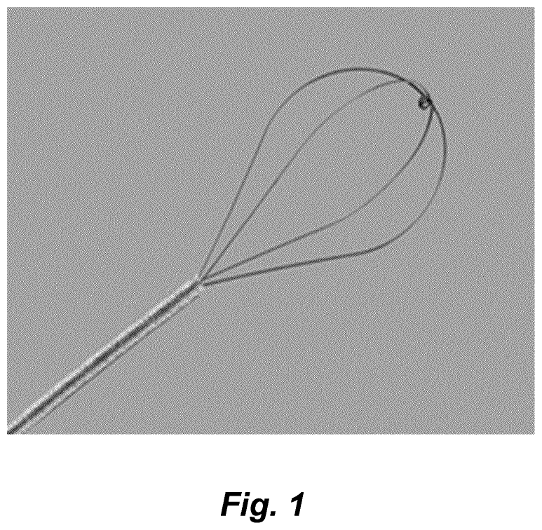

With the intubated animal in the supine position, a pliable wire-basket (NCircle® Nitinol Tipless Stone Extractor NTSE-022115-UDH, Cook Medical; FIG. 1) was fed in the retracted position down the ET tube to approximately the carina (based on an average trachea length measurement). The basket was extended, fully expanded, and drawn up and down the trachea to the carina (approximately 15 times) for 30 seconds followed by withdrawal from the lung. Two 25 μL aliquots of LV-LacZ (2×10⁹ TU/mL) were delivered via the ET tube ten minutes after the perturbation event to allow time for the animals' respiration to return to normal following the procedure.

Bioluminescence Imaging (BLI)

Either 1-week or 12-months following LV-FLAG-Luc-GFP delivery rats were anaesthetised with 2.5% inhaled isoflurane. Once unconscious, 200 L of 15 mg/mL D-luciferin (Cayman Chemicals, USA) was delivered to the nostrils as a bolus. The animal was imaged in the supine position (IVIS Lumina XRMS in vivo imaging system, PerkinElmer, USA) while maintained with 1.5-2% isoflurane. Animals were imaged with an automatic exposure at 5-minute intervals for up to 15 minutes post D-luciferin delivery. The animal was then humanely killed with IP delivery of sodium pentobarbital (150 mg/kg). The trachea and lungs were immediately excised and imaged separately in a petri dish containing PBS. The resulting bioluminescence flux (photons/second) was measured using the contour tool (Living Image, version 4.7.2, PerkinElmer, USA).

Assessment of LacZ Gene Expression

Animals were humanely killed and the airway tissues harvested for LacZ staining. Tissues were fixed in 4% PFA and stained for β-galactosidase (LacZ) activity using X-gal as previously described (Cmielewski, P., et al. (2017) Exp Lung Res 43(9-10): p. 426-433). Following X-gal staining, the trachea was cut open longitudinally, and separated into two halves. En face images of the tissue were acquired with a Nikon SMZ1500 stereo microscope, DigiLite 3.0 MP Camera and TC capture software (Tucsen Photonics, China). Illumination intensity was not altered between samples. Tracheas were subsequently paraffin-embedded, sectioned at 5 μM and counterstained with nuclear fast red. Histology images were captured with a Nikon Eclipse E400 microscope, DS-Fi2-U3 camera and NIS-elements D software (v5.20.00).

Digital Quantification of LacZ Staining in En Face Trachea Images

The amount of LacZ staining in the enface images of the trachea was quantified using a custom-written script in Matlab (R2010a, MathWorks) that calculated the area of the trachea that was the specific shade of blue/green that matched the LacZ-positive cells. Briefly, images were converted from the RGB to HSV colour space, and separate thresholds were applied to the hue (0.45<H<0.6) and saturation (s>0.4) channels. The value channel was not used because the illumination across the sample was uneven and did not help discriminate the transduced cells. A binary mask was created from the regions of the image that satisfied both criteria. The mask area was converted from pixels into mm.

Statistical Analyses

Statistical analyses were performed using either R v4.0.0 [23] or GraphPad Prism v8 (GraphPad Software, Inc.). For the short-term study, comparisons of flux between PBS and LPC groups were performed using Welch two sample t-tests on log-transformed data. To compare flux between the PBS and LPC treatments for the long-term study, a hurdle regression model was fitted using the hurdle function in the pscl package in R (Zeileis, A., Kleiber, C., and Jackman, S., (2008) J Stat Softw 27(8)). The hurdle regression model is a two-part model; a binary logit model to distinguish between the positive counts and those below the detection limit (assigned the value 100), and a zero-truncated negative binomial model for the positive counts. Treatment (PBS, LPC) was fitted as the regressor in both parts of the model. Differences between the groups were assessed using a Wald Chi-Squared test. The LacZ staining area measurements from the en face images were analysed using a one-way ANOVA with Tukey's multiple comparisons test.

Results

Luciferase Gene Expression Assessed 1-Week Post Gene Transfer

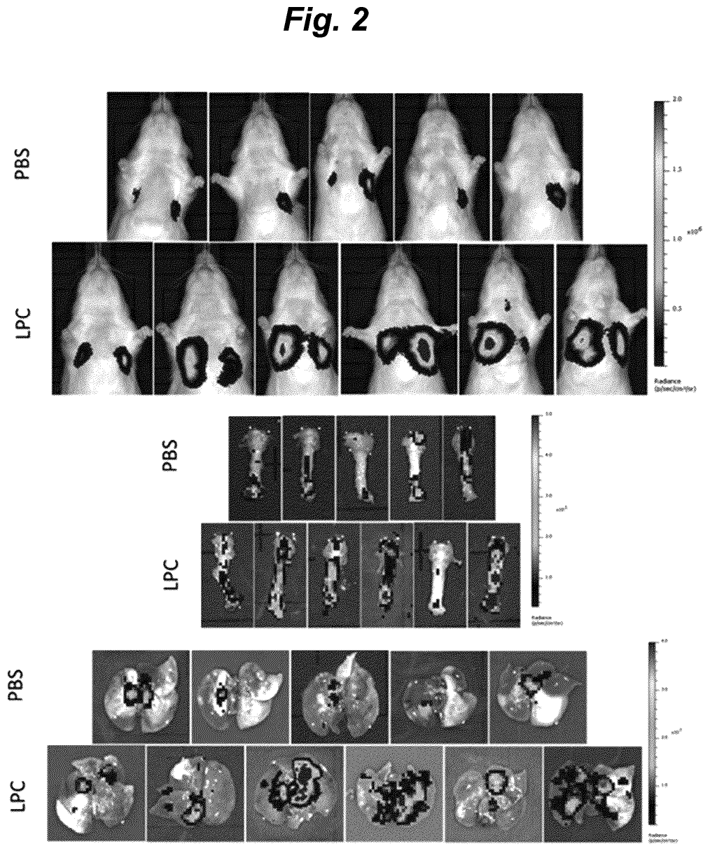

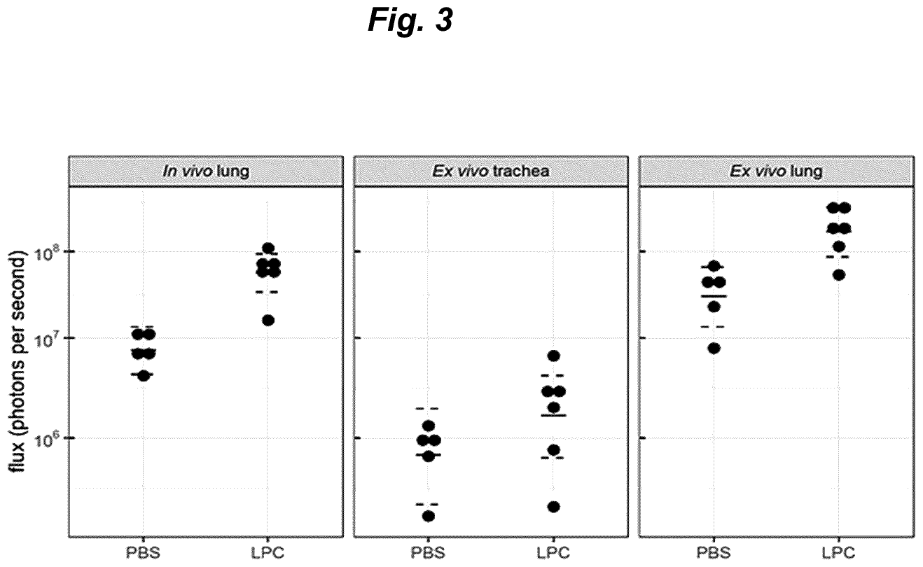

In the short-term assessment, rats received either PBS or LPC conditioning and were imaged 1-week following a single LV gene delivery (FIG. 2). In vivo BLI revealed that LPC conditioning resulted in significantly higher flux when compared to animals that received the PBS sham treatment (FIG. 3). Bioluminescence was predominantly observed in the lungs, with only one animal exhibiting signal within the trachea region. Upon excision of the tissues and subsequent imaging, bioluminescence was observed in the trachea of all animals. Ex vivo data revealed that there was no difference in flux between the LPC and PBS conditioned groups for the trachea, while the excised lungs revealed that LPC conditioned rats had significantly greater flux levels, consistent with the in vivo data. Adjusting for sex in the modelling did not result in differences from the unadjusted models.

Luciferase Gene Expression Assessed 12-Months Post Gene Transfer

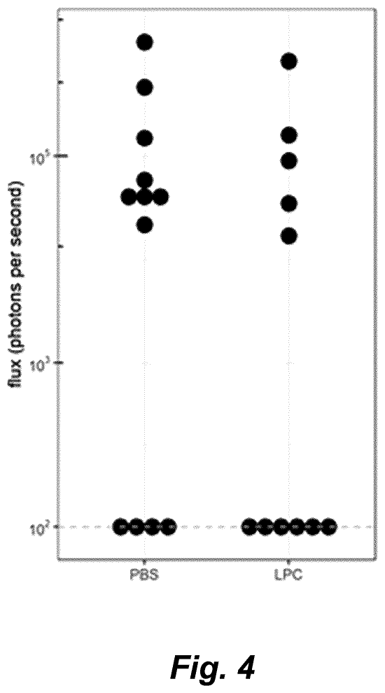

In the long-term study assessing chemical conditioning, few animals from either the LPC or PBS group had detectable levels of in vivo bioluminescence at the 12-month time point, therefore statistical comparisons could not be performed on this data. Ex vivo imaging of the trachea also revealed insufficient animals with bioluminescence present to perform statistical analysis. Imaging of the ex vivo lungs showed that close to half the animals from both LPC and PBS groups had values recorded at the limit of detection. Additionally, the flux levels measured in the ex vivo lungs at 12 months were substantially lower compared to the animals imaged at 1-week post-delivery. There was no significant difference in ex vivo lung flux between LPC conditioned and PBS animals 12-months following LV-delivery (FIG. 4).

Tracheal LacZ Staining Following Chemical Conditioning and Physical Perturbation

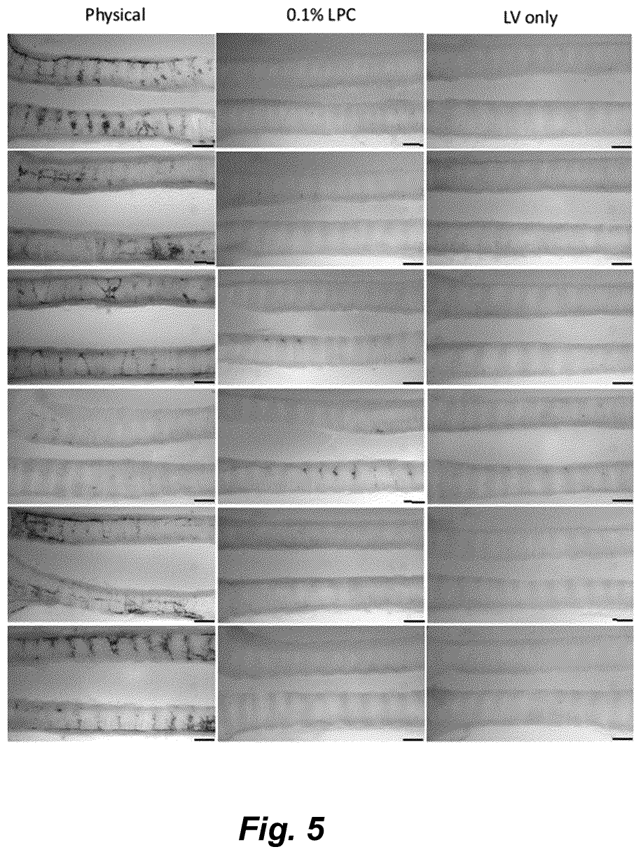

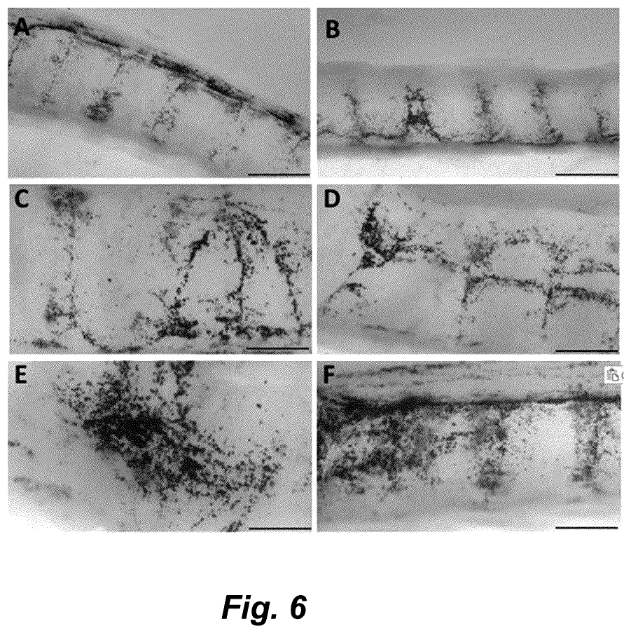

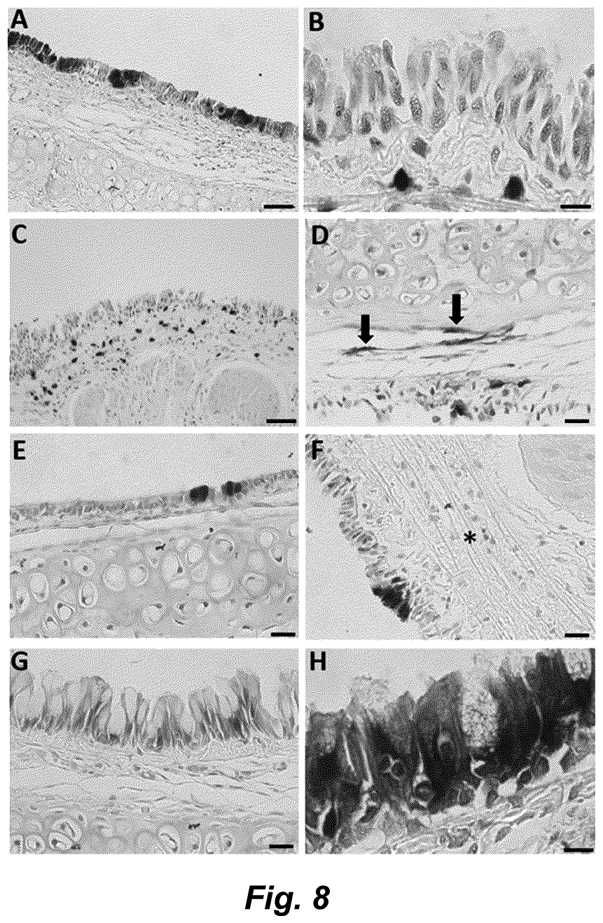

Enface observations of the trachea revealed varying levels of LacZ staining as a result of the perturbation method used (FIG. 5). Visually, rats that received physical perturbation prior to LV-delivery demonstrated greatly enhanced LacZ staining in the trachea when compared to rats that received LPC conditioning or LV vector only. Interestingly, one animal in the physical perturbation group exhibited much lower staining levels compared to the others, indicative of potential variability with this technique. The transduction patterns observed as a result of physical perturbation were not uniform in appearance. Striated regions of increased staining over the intercartilaginous ligaments were apparent in all specimens, possibly reflecting the presence of cilia-rich zones that overlay the ligament segments and preferential transduction of ciliated cells by the LV gene transfer vector. Varying LacZ staining patterns and strong expression were observed in animals that received physical perturbation (FIG. 6). Notably, rats in the LPC and LV only groups demonstrated the highest levels of staining in the proximal trachea (FIG. 8), likely due to unintentional ET tube-induced epithelial damage in this region, providing further support for the effectiveness of physical perturbation in boosting airway gene transfer. En face LacZ staining was also observed in the lungs of all specimens, however, this tissue was not analysed because the physical perturbation was only applied to the trachea, thus comparisons of lung transduction could not be performed between groups.

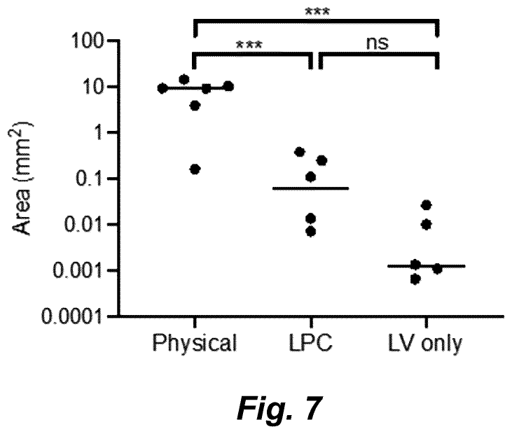

Digital quantification of the blue-green coloured regions in the enface trachea images demonstrated significantly greater levels of LacZ staining in the physical perturbation group, when compared to LPC conditioned and LV vector only control rats (FIG. 7). The physical damage group demonstrated more than a 1000-fold increase in the area of LacZ staining when compared to the control group. There was no statistically significant difference in staining area between LPC conditioned rats and controls that received only LV vector with no epithelial perturbation prior to delivery.

Histological Observations Arising from Physical Perturbation

A range of different cell types were transduced as a result of performing physical perturbation prior to LV vector delivery (FIG. 8). Based on morphological appearance and location within the tissue, LacZ-positive ciliated, non-ciliated, and basal cells were observed. Cells within the lamina propria were also transduced, including suspected fibroblasts and possible macrophages. The tracheal tissue demonstrated evidence of repair processes caused by damage to the epithelium and surrounding regions. Within the lamina propria there were regions of connective tissue proliferation, consisting of collagen formation and fibroblasts. Most regions of the tracheal epithelium had fully regenerated by 1-week post treatment. However, occasionally areas of epithelium were found to still be undergoing repair, as indicated by an attenuated appearance, lack of cilia, and rounding of the columnar cells. Goblet cell hyperplasia was also apparent at the carina and upper bronchi of two animals. Interestingly, many of these hyperplastic goblet cells appeared to be LacZ-positive.

Discussion

Gene-addition therapy is a promising option for treatment of certain pulmonary diseases, but there are still many challenges to successful translation to the clinic. Delivery of therapeutic agents to cells of the airway epithelium remains one of the greatest challenges to achieving effective lung-based genetic therapies, including gene-addition and gene editing strategies. The lung has evolved mechanisms to resist invasion by foreign bodies, with the airway epithelium acting as a physical barrier that significantly reduces the efficacy of airway gene transfer. Many viral vectors used for airway gene therapies have a natural tropism toward receptors located on the basolateral membrane and thus produce limited transduction via the apical surface.

To overcome inefficient transduction, strategies have been employed to disrupt the integrity of the airway epithelium prior to gene delivery, allowing the vector access to basolateral membrane-located receptors and to cells that are not in direct contact with the airway lumen such as basal cells. The work reported here employed normal rats to develop and assess chemical and physical airway perturbation methods. In these studies, the trachea was used as surrogate for lower airways, as it is easily accessible for physical perturbation techniques and the cellular architecture is similar to human airways.

In this work, LPC conditioning was assessed for the first time in rat airways. In this study, gene expression levels were found to be significantly increased in the lungs of rats that received LPC conditioning when assessed 1-week post vector delivery. Upon in vivo imaging, only one animal exhibited bioluminescence signal within the trachea, but following excision of the tissues and subsequent imaging, bioluminescence was observed in the trachea of all animals, albeit at varying levels. When imaged ex vivo the flux emitted from the trachea was substantially lower than the ex vivo lung, therefore the low bioluminescence in combination with the need to detect the signal through the overlying skin and tissue may explain the lack of tracheal bioluminescence upon in vivo imaging. Notably, when the trachea was excised and imaged, LPC did not appear to enhance bioluminescence flux when compared to the PBS-sham group.

Long-term gene expression was assessed in a separate cohort of rats, which revealed that luciferase gene expression persisted in the lungs of some animals for up to 12 months following a single LV-delivery, irrespective of whether LPC airway conditioning was performed. These two studies indicate that while LPC conditioning initially improved gene transduction levels in the lungs, it did not increase the longevity of gene expression, suggesting that stem cell transduction was not substantially enhanced with use of LPC. It is suspected that stem cells were transduced in animals from both LPC and PBS-sham groups, as the duration of gene expression exceeds the four month lifespan of terminally differentiated tracheal-bronchial epithelium in adult rodents.

While LV-mediated gene transfer demonstrated persistent luciferase gene expression in a proportion of rats, many individuals from both the LPC conditioning and PBS-sham groups had undetectable flux by 12 months. It remains unclear why some individuals responded better than others, though this phenomenon is consistently observed in airway gene therapy animal studies. Possible factors may include but are not limited to, the proportion of stem cells transduced, varying host immune responses to the vector and formulation, size of the animal, which dictates the surface area to vector volume ratio, variable distribution of LPC and vector within the airways, and the amount of time that the vector is in contact with the airway surface.

In the separate study here employing LV-LacZ vector, physical perturbation of the airway epithelium demonstrated very high levels of gene transduction in the trachea when compared to animals receiving LPC chemical conditioning or LV vector only. Moreover, LPC conditioning did not significantly enhance tracheal LacZ expression above the control (LV only) group, consistent with the short-term bioluminescence study where LPC use did not result in a significant increase in tracheal flux when compared with the PBS-sham group. However, it is important to note that the LPC conditioning protocol used in these studies had been previously established for mice, therefore the concentration or volume employed may not be optimal for rats.

As with many viral receptors, the VSV-G receptor (low-density lipoprotein) is located on the basolateral surface, and transduction via the apical surface is typically low level. While the mechanisms here are not fully understood, it is likely that physical perturbation increases gene transfer via two potential routes: (1) disruption of the integrity of the epithelial tight-junctions allowing the gene transfer vector entry to epithelial cells via basolateral receptors, and/or (2) removal of transduction-resistant columnar cells and exposure of more susceptible cell types on the basement membrane (e.g. basal cells). In this study, physical damage was found to produce basal cell transduction. Both enface and histological examination revealed distinct clusters of LacZ-positive cells (e.g. FIG. 6E). These clusters suggest that one or more basal cells have been transduced and subsequently differentiated into a LacZ expressing pseudostratified epithelium, consistent with previous studies showing the timing of epithelial regeneration in rat airways following mechanical injury. Alternatively, clusters of LacZ-expressing cells may be due to strong focal damage or repetitive contact with the basket, making these areas more susceptible to LV-transduction.

Physical perturbation may provide a newly-applied method to boost access to basal cells. An airway gene-addition therapy that transduces only the terminally differentiated surface cells will result in an inevitable waning of transgene expression with normal cell turnover, and the need for vector re-administration. While it is unlikely to be completely avoided, repeated administration of viral-based gene therapies is undesirable as immune responses may be elicited upon subsequent deliveries, reducing the efficacy with each dose. Basal cells are multipotent stem cells of the conducting airways and have the capacity for self-renewal, clonal expansion, and the ability to differentiate into epithelial cells types to maintain homeostasis and repair following injury. Successful transduction of basal cells has the ability to produce enduring gene expression, as the gene-corrected progeny will repopulate the surface epithelium following natural cell turnover. It is therefore desirable to develop an airway gene-addition therapy that not only produces efficient gene transfer but can also transduce basal cells.

In some regions of the trachea, cells within the lamina propria appeared to be transduced. While a certain degree of epithelial injury was expected, these findings suggest that in some areas the physical perturbation extended too far into the airway surface, likely due to forces produced by the wire basket or repetitive contact. Goblet cell hyperplasia was also noted in some animals, a protective response that can occur as a result of mucosal irritation. Notably, the animals that displayed goblet cell hyperplasia had many LacZ-positive goblet cells within these regions, suggesting that intact basal cells were transduced and subsequently differentiated into goblet cells upon epithelial regeneration. Our previous short-term assessments have shown that VSV-G pseudotyped LV vectors rarely transduce goblet cells, so their presence further indicates likely expansion and differentiation of transduced basal cells.

One major benefit of physical perturbation noted in this study is the ability to achieve high transduction levels from a relatively small number of LV particles per animal (1×10⁸ TU). By using epithelial disruption methods to improve gene transfer efficacy, the LV titres required to achieve therapeutic gene correction are reduced, thus improving the feasibility of an in vivo gene therapy approach. Moreover, such reductions in vector titre would provide the benefits of lower LV production time and cost, as well as lessened immune responses. The advantages of physical perturbation could also be applied to improving the efficacy of other lung-based genetic therapies including delivery of gene editing machinery, nanoparticle-based gene transfer systems, and stem cell transplantation strategies. For instance, airway engraftment of transplanted cells could be facilitated via use of physical perturbation rather than chemical agents, which have previously been employed preclinically to achieve epithelial disruption, but have disadvantages such as toxicity and poor delivery control.

While the tracheal physical perturbation study performed here shows promising proof-of-principle data, the target for CF airway gene-addition therapy will be the lower large-medium sized airways, as this is where disease pathophysiology occurs. Further work is needed to assess the feasibility of this approach in lower airways of rats, with our previously established rat bronchoscopy methods offering a potential option for enabling access to these small airways for perturbation (McIntyre et al (2018) Hum Gene Ther Methods 29(5): p. 228-235).

In summary, although LPC conditioning initially increased gene expression levels in rat lungs, it did not appear to improve reporter gene expression longevity. Even without conditioning, a proportion of rats had measurable luciferase flux present in the lungs 12 months following dosing, indicating that a single LV delivery can provide persistent gene expression, albeit at low levels.

On the one hand, physical perturbation to the trachea immediately prior to gene transfer resulted in a 1000-fold increase in LacZ staining when compared to animals that did not receive any airway perturbation, demonstrating that this approach can provide significant benefit for improving gene transfer efficacy and warrants further development and investigation.

### Example 2—Immediate Physical Effects of Epithelial Cell Perturbation Methods

Studies were undertaken to assess the immediate physical effects of epithelial cell perturbation methods (trachea removed and fixed −10 mins after use; 2-3 rats per technique)

The control for the studies was unperturbed tracheal epithelium. The following treatments were uses to perturb the tracheal epithelium:
Directed fluid, 0.5 mm orifice stream (ex-vivo)
Wire baskets, two types (in vivo)—Cook Medical NCircle, NGage
Brush (in vivo)
Piksters Interdental #3
Balloon catheter (in vivo)—Olympus
Wire loops (in vivo)—Olympus
Biopsy Forceps (used expanded, once placed in vivo)—Storz
(1) Control Studies

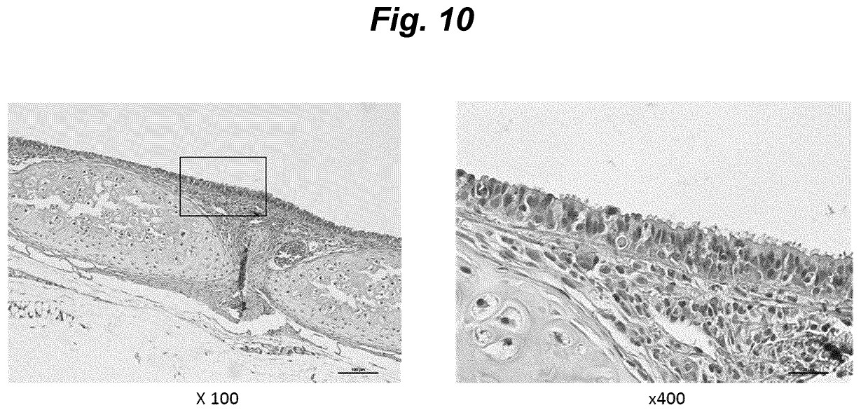

The histology of normal rat tracheal epithelium (H&E-stained), without perturbation is shown in FIG. 10.

(ii) Effect of Directed Fluid Stream onto Airway Surface

Introduction

The effect of disturbance of tracheal airway epithelium in fresh ex-vivo rat tracheas was examined after a targeted fluid disruption using a microfocussed stream of fluid.

Methods

A consumer-level Waterpik dental cleaning device was used, and employed room temperature PBS. The device utilized the 0.5 mm orifice wand tip.

Fresh ex-vivo rat trachea was opened longitudinally along one surface and pinned out in Sylgard dish, and drained just prior to the Waterpik device use. The stream was hand-directed under stereomicroscope vision at various force settings. Once completed (<5 seconds) the trachea was unpinned and fixed in 10% NBF for standard histological processing.

Results

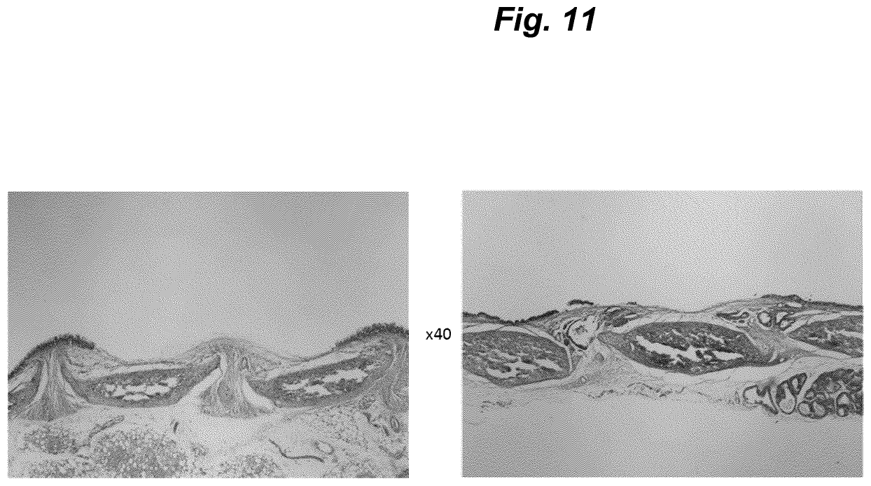

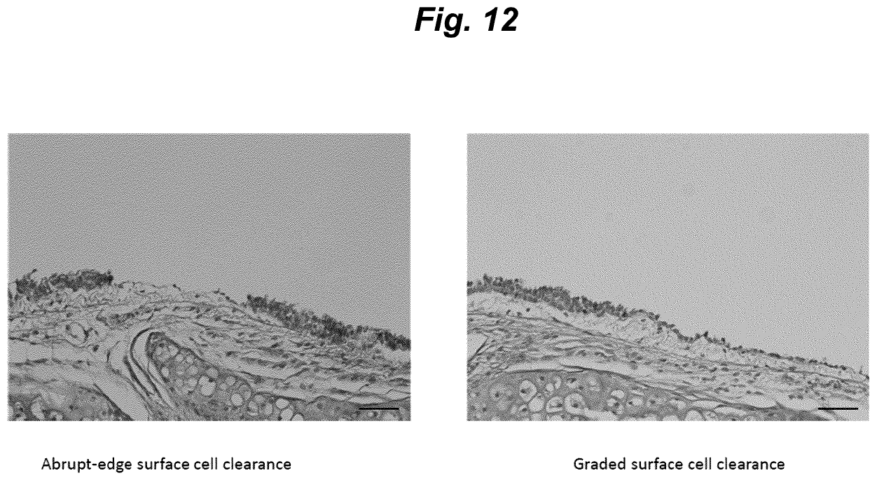

The fluid stream produced cleanly-denuded regions of the airway surface (see FIGS. 11 and 12)). Both abrupt-edge and graded-edge surface-cell removal effects were noted. At high magnification the retention of the basement membrane and basal cells occurred in some regions, while in other regions (not shown) these were removed.

Discussion

These findings show the effectiveness of a targeted fluid stream in removal of surface cells of the airway epithelium. A range of effects from mild to severe were observed, an expected finding due to the lack of distance and force control in this proof of principle study.

(iii) Airway Disturbance Effects Produced by a Cook “N-Circle” Wire Basket

Introduction

The effect of disturbance of tracheal epithelium in an anaesthetised rat using a pliable wire basket with a tapered tip was examined

Methods

The rat was anaesthetised and endotracheally-intubated supine. The wire basket (NCircle® Nitinol Tipless Stone Extractor NTSE-022115-UDH, Cook Medical) was fed down the endotracheal tube, expanded, and was moved up and down the trachea for 30 seconds. The procedure took approximately 30 secs to complete.

Ten minutes later the animal was humanely killed, and the trachea harvested and processed for histology.

Results

The clear surface disturbance and localised surface cell removal was apparent in H&E-stained sections of the trachea.

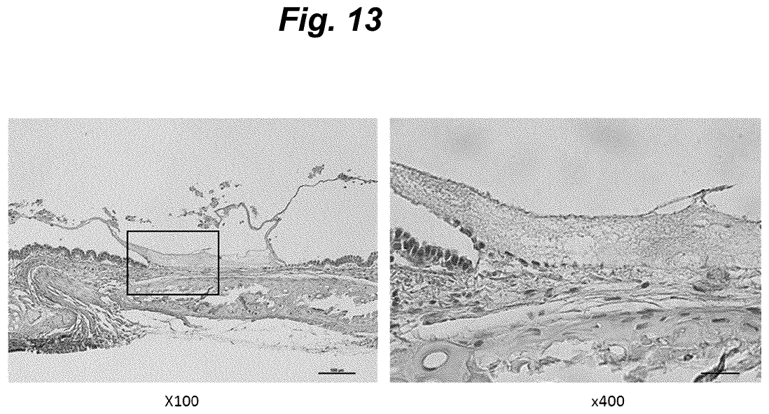

The results are provided in FIG. 13 and show basement membrane and basal cells of the epithelial layer were retained or removed depending on the position, however in most regions the basement membrane and basal cell layer was retained. A fluid exudate and removed cells overlays the surface. The cell removal effects were present along the length of the treated tracheal regions.

Discussion

This device is highly pliable when expanded and is readily moved along the airway, allowing for simple placement and motion in the airway and for cell disruption along most of the targeted airway region.

(iv) Airway Disturbance Effects Produced by a Cook “N-Gage” Wire Basket

Introduction

The effect of disturbance of tracheal epithelium in an anaesthetised rat using a pliable wire basket with an umbrella-like front wire face was examined.

Methods

The rat was anaesthetised and endotracheally-intubated supine. The wire basket (NGage® Nitinol Stone Extractor, Cook Medical) was fed down the endotracheal tube, expanded, and was drawn up the trachea over 30 seconds.

Ten minutes later the animal was humanely killed, and the trachea harvested and processed for histology.

Results

The clear surface disturbance and localised surface cell removal was apparent in H&E stained sections of the trachea.

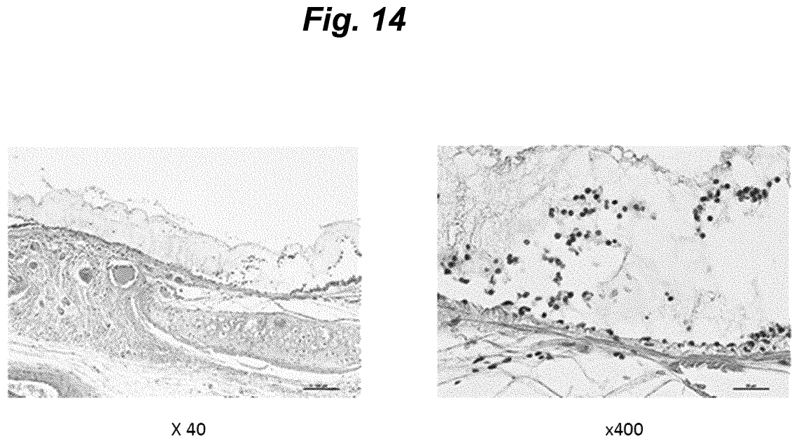

FIG. 14 shows that the basement membrane and basal cells of the epithelial layer could be retained with this procedure, but in other regions the basement membrane was breached. A fluid exudate and removed cells overlays the airway surface. Cell removal effects were present at these varied levels along the length of the treated tracheal regions.

Discussion

This device incorporates a distal umbrella-like face that extended the physical ‘touch’ of the airway surface more robustly and circumferentially than the other wire baskets used

(iv) Effect of Brush Perturbation of Airway Surface

Introduction

The effect of disturbance of tracheal airway epithelium in live anaesthetised rats was examined after a disruption using a miniature dental brush.

Methods

A Pikster interdental brush Size 3 was used, with the wire base removed from its plastic holder and bonded to a fine thin stiff wire, for placement via the endotracheal tube.

The rat was anaesthetised and endotracheally-intubated supine. The brush was fed down the endotracheal tube, and then drawn up and down the trachea for −30 seconds then withdrawn. The procedure took approximately <30 secs to complete. Ten minutes later the animal was humanely killed, and the trachea harvested and processed for histology.

Results

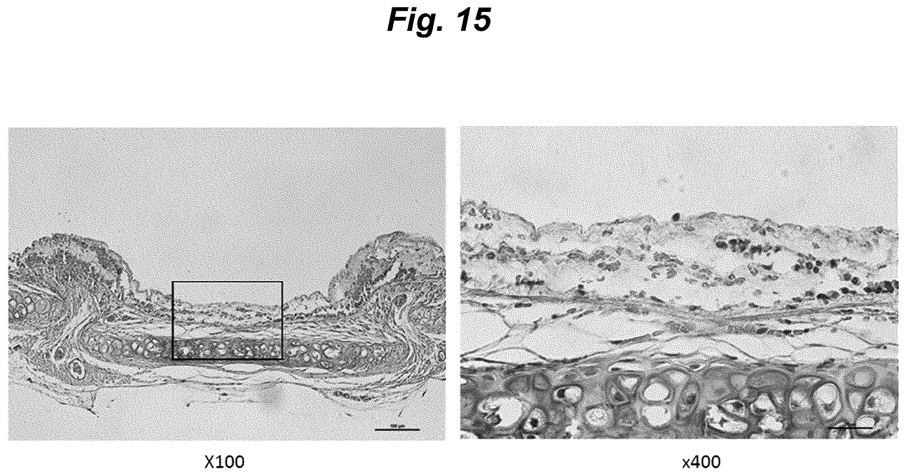

The brush produced broad regions of denuded airway surface (see FIG. 15).). A fluid exudate and removed cells overlays the airway surface. The cell removal effect was more completed than many other methods, nevertheless in some areas basement membrane and basal cells were retained while in others the basal cells and basement membrane were lost.

Discussion

These results show that fine brushes can be used to reliably denude surface cells from affected regions of the airway epithelium in a controlled fashion via an endotracheal tube.

(vi) Effect of Balloon Tip Perturbation of Airway Surface

Introduction

The effect of disturbance of tracheal airway epithelium in live anaesthetised rats was examined after a disruption via a balloon-tipped catheter.

Methods

An Olympus bronchoscopic balloon catheter was used.

The rat was anaesthetised and endotracheally-intubated supine. The balloon catheter was fed down the endotracheal tube, the balloon expanded, and then drawn up the trachea for ˜30 seconds; after collapsing the catheter was then withdrawn. The procedure took approximately <30 secs to complete. Ten minutes later the animal was humanely killed, and the trachea harvested and processed for histology.

Results

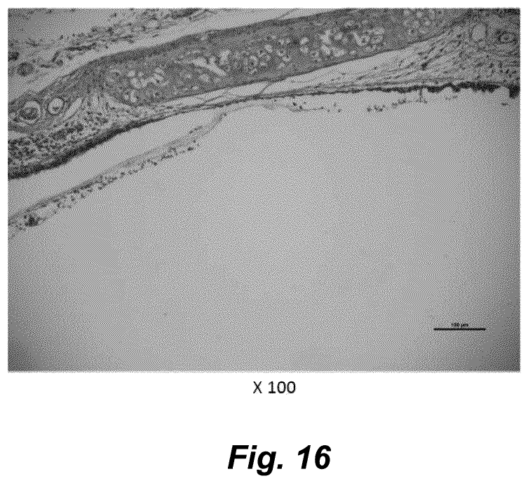

The expanded balloon produced broad regions of denuded airway surface (see FIG. 16). A fluid exudate and removed cells overlays the airway surface here. Areas where basement membrane and basal cells were retained, as well as some less frequent areas these were lost, were observed.

Discussion

These findings show that an expandable balloon tip can remove surface cells from affected regions of the airway epithelium in a controlled fashion.

(vii) Effect of Wire Loop Perturbation of Airway Surface

Introduction

The effect of disturbance of tracheal airway epithelium in live anaesthetised rats was examined after disruption using a wire loop.

Methods

An Olympus wire loop (SD-221U-25 Crescent Electrosurgical Snare) was used.

The rat was anaesthetised and endotracheally-intubated supine. The collapsed wire loop was fed down the endotracheal tube, expanded, and was drawn up and down the trachea for 30 seconds. The procedure took approximately 30 secs to complete. Ten minutes later the animal was humanely killed, and the trachea harvested and processed for histology.

Results

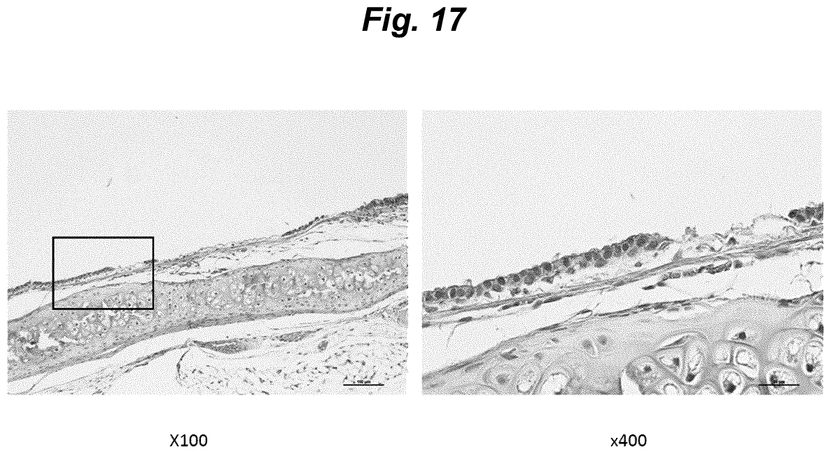

The loop produced regions of denuded airway surface that were small (see FIG. 17) and not widely distributed. At high magnification the basement membrane and basal cells were retained in most regions, although in some regions these were removed.

Discussion

These findings show that a wire loop can produce controlled removal of surface cells of the airway epithelium.

(viii) Effect of the Perturbation of the Airway Surface Via the Outer Surface of Biopsy-Forceps

Introduction

The effect of disturbance of tracheal airway epithelium in live anaesthetised rats was examined after disruption using the smooth outer edges of expanded bronchoscopic biopsy forceps.

Methods

Storz bronchoscopy forceps were employed.

The rat was anaesthetised and endotracheally-intubated supine. The biopsy forceps were fed down the endotracheal tube, the jaws opened, the opened forceps drawn up the trachea; the jaws were then closed, the device moves to the distal end of the trachea, and the process repeated several times over 30 seconds. The procedure took approximately 30 secs to complete. Ten minutes later the animal was humanely killed, and the trachea harvested and processed for histology.

Results

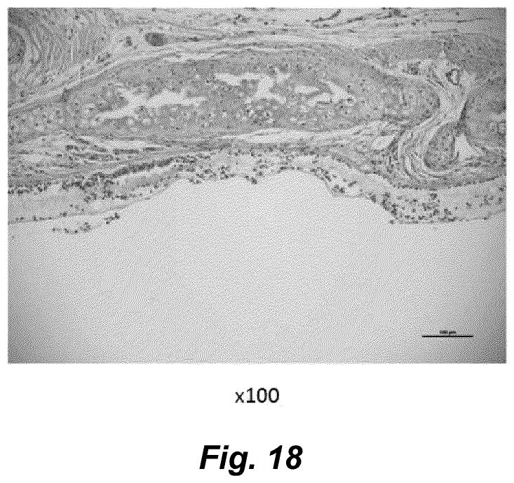

The outer surface of the forceps produced regions of denuded airway surface along the trachea that showed clear regions of surface cells removal. FIG. 18 shows a denuded region, covered with an exudate of fluid and removed cells. At high magnification (not shown) the basement membrane and basal cells were typically retained in most regions.

Discussion

These findings show that bronchoscopic biopsy forceps, used for their smooth jaw outer surfaces when expanded, can produce controlled removal of surface cells of the airway epithelium.

### Example 3—Simultaneous LV-LacZ Vector and Physical Airway Disturbance (Cook “N-Gage” Wire Basket)

Introduction

The effect of simultaneous (perturbation and delivery being contiguous with the procedure) disturbance of tracheal epithelium using a pliable wire basket with delivery of a lentiviral (LV) LacZ gene vector was tested in an anaesthetised rat for its ability to produce in-vivo airway cell LacZ transduction.

Methods

The rat was anaesthetised and endotracheally-intubated supine. The wire basket (Cook N-Gage) was fed down the endotracheal tube, expanded, and was drawn up the trachea over 30 seconds, followed by immediate delivery of LV-LacZ vector (2×25 μL aliquots) into the trachea via the endotracheal tube using a fine extended pipette tip. The entire procedure took approximately <1 minute to complete.

One week later the animal was humanely killed and the trachea was harvested and processed for LacZ transduction (X-gal stain)

Results

Surface Disturbance Alone Produced No Transduction, and Use of the LV-LacZ Vector Alone Produced Scattered Low-Level Cell Transduction

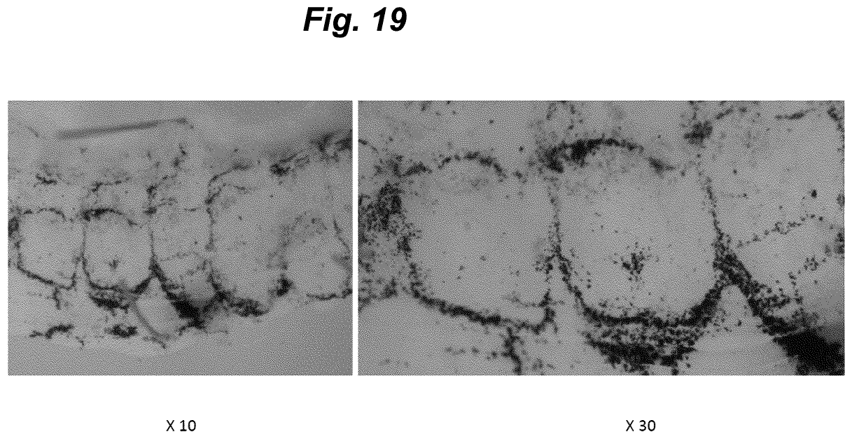

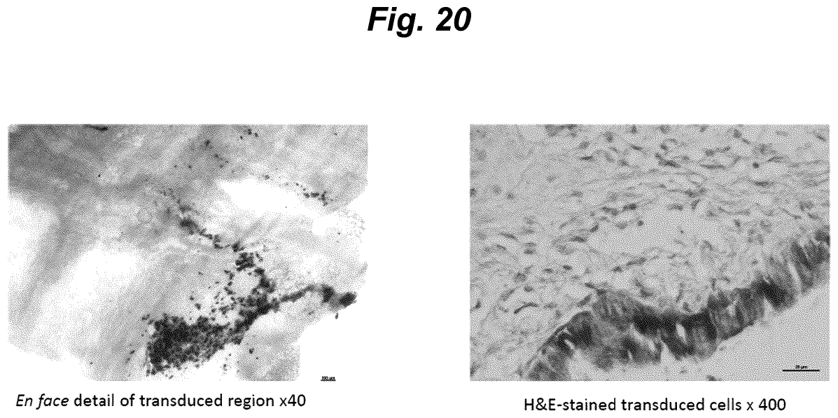

FIG. 19 (en face stereomicroscope image of opened airway) shows the extensive LacZ transduction (black regions being the blue XGal—stained cells) that resulted from simultaneous airway disturbance and vector delivery. A 40× magnification en face view, and a ×400 magnification of a cross section of transduced epithelium of stained regions are provided in FIG. 20. The staining pattern (FIG. 19) reveals the regularity of effect on each tracheal segment region, with predominantly axial lines of transduced cells resulting from epithelial cell surface disturbance caused by the axial transit of the pliable fine wires of the wire basket. The expanded wires of the NGage device are more transversely arranged and are likely to provide the more circumferential transduction seen here.

Discussion

This finding validates the effectiveness and simplicity of the simultaneous disturbance/vector-delivery technology and protocol. Gene delivery has occurred along the length of the disturbed tracheal area, providing extended and controlled transduction across the targeted region. Such orderly and broad transduction coverage has not been reported using any other delivery technique in live animal airways.

### Example 4—Effect of LV-LacZ Vector Delivery 10 Minutes after Cook “N-Gage” Wire Basket Airway Disturbance

Introduction

The effect of disturbance of tracheal epithelium in an anaesthetised rat using a pliable wire basket was tested for its ability to produce LacZ reporter-gene transduction of airway cells using a lentiviral (LV) vector.

Methods

The rat was anaesthetised and endotracheally-intubated supine. The wire basket was fed down the endotracheal tube, expanded, and was drawn up the trachea for 30 seconds. Ten minutes later LV-LacZ vector (2×25 μL aliquots) was delivered into the trachea via the endotracheal tube using a fine extended pipette tip. Control animals received only LV-LacZ delivery.

One week later the animal was humanely killed and the trachea was harvested and processed for LacZ transduction (X-gal stain)

Results

The use of the LV-LacZ vector alone produced rare and scattered cell transduction.

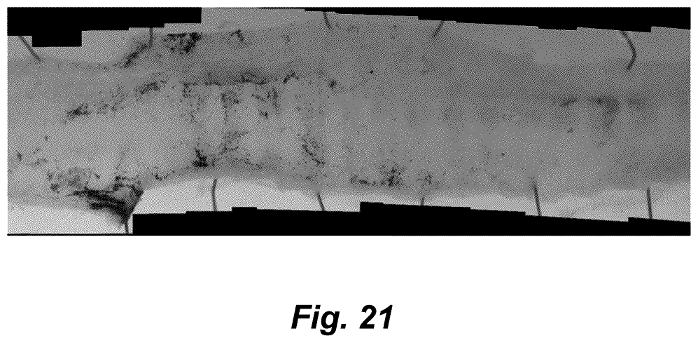

FIG. 21 (a longitudinally-stitched en face image from a number of stereomicroscope images of the airway) showed extensive LacZ transduction after the airway disturbance—vector delivery procedure. X-gal staining was often associated with the tracheal segment regions, resulting from epithelial cell surface disturbance caused by the axial transit of the pliable fine wires of the wire basket.

Discussion

This finding indicates that a short delay in vector delivery does not substantially alter the effectiveness of the procedure for boosting reporter gene transduction from a LV-LacZ vector across the targeted region, when compared to the simpler simultaneous wire cage disruption/vector-delivery procedure described previously herein.

### Example 5—Devices for Delivering Agents to the Respiratory System

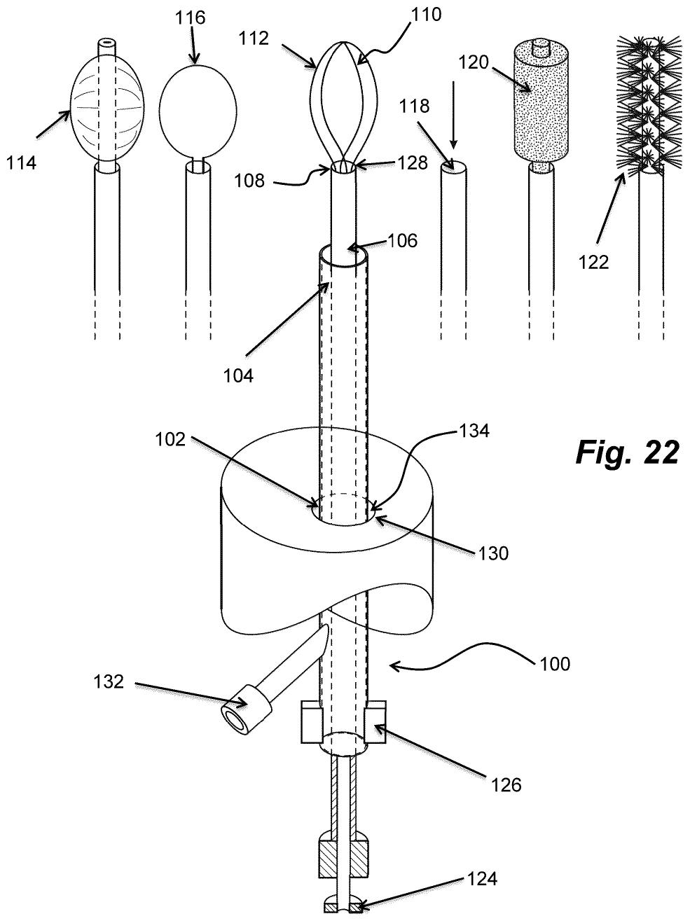

A representation of a device 100 for perturbing the airway and delivering an agent to the perturbed airway according to one embodiment is shown in FIG. 22.

The device 100 is configured for insertion into the airway 102, namely, the trachea, the bronchus and the bronchioles of a subject, and the size of the device adapted accordingly. In this regard, the device 100 may be of a size that permits it to extend to a number of branch levels down into the airway. The device 100 is able to be manually advanced into the airway, or to be manually withdrawn from the airway 102, by forward or backward movement respectively of the device 100.

The device comprises an outer tube 104, which encompasses an inner tube 106 which is itself moveable within the outer tube 104. The inner tube comprises, at its distal end 108, an end component 110 for perturbing the surface of the airway when deployed. In one embodiment, the end component 110 is a cage 112 made of a material such as a flexible and resilient metal or plastic, although in other embodiments the end component 110 may be an inflatable balloon 114, a flexible and resilient loop 116, a suction device 118, a sponge 120 or a brush 122. The end component 110 is configured so that when the inner tube 106 is located within the outer tube 104, the end component 110 is held in a collapsed or retracted configuration ready for deployment so as to be used at the selected site in the airway when desired.

Typically, the device 100 is manually manoeuvred into the airway and advanced to a suitable point of the airway 102 for perturbation (for example by using a scope), utilising the thumb rest 124 and the finger stops 126 to advance the inner tube 106 upon application of pressure. During insertion of the device 100 into the airway, the inner tube 106 is positioned so that the end component 110 is accommodated within the outer tube 104 and thus in a compressed configuration for cage 122, loop 116, sponge 120 or brush 122. For balloon 114 and suction device 118, these end components are held inside outer tube 104 awaiting use.

In the embodiments utilising end components 112 (flexible cage), 116 (flexible loop), 120 (compressible sponge) and 112 (compressible brushes), the end component 110 is compressible to permit location of the end component 110 in the outer tube 106 prior to use. When the end component 112, 116, 120, or 112 is to be deployed, the inner tube 106 is moved forward allowing the end component 112, 116, 120 or 112 to move past the end 128 of outer tube 106 and expand so as to contact the inner surface 130 of the airway 102 and cause some form of physical disruption, particularly when the inner tube 106 is moved relative to the airway surface 130. After use of the end component 112, 116, 120 or 122 to perturb the surface, inner tube 106 is retracted into outer tube 104, and the end components 112, 116, 120 or 122 compressed so as to be accommodated within outer tube 104. As described previously herein, the device may permit adjustment of the size of the end component when expanded and/or the amount of pressure that can be applied to the airway surface

In the embodiment utilising end component 118, being a suction end 118, the end component is located in the outer tube 106, and then the end component is advanced past the end 128 of outer tube 106 so that suction can be applied to the airway. The suction applied can be used to apply a negative pressure to the airway and thereby physically disrupt the surface of the airway. Once perturbation has been completed, suction may be terminated and the suction component 118 retracted into the outer tube 104.

In the embodiment utilising end component 110, being an inflatable end 114, the end component is located in the outer tube 106 in a deflated configuration, and then the end component is advanced past the end 128 of outer tube 106 so that the balloon can be inflated, for example with a gas or liquid. The increase in size of the balloon can be used to apply a controlled distensive force to the airway and thereby physically disrupt the surface of the airway. Once perturbation has been completed, the balloon may be deflated and the balloon 114 retracted into the outer tube 104.

The device 100 further utilises an entry port 132 to introduce the agent into the device 100 before, during and/or after perturbation of the airway has been conducted. In this case, the entry port 132 permits the agent to be introduced into the device 100 and to travel to the end of the device 128 along the gap 134 formed between the outer tube 104 and the inner tube 106, thereby delivering the agent to the area where the surface has been perturbed. For example, the agent may be delivered using a method such as lavage, atomisation, nebulisation, aerosolization, direct instillation, nasal administration or fluidic administration. The agent may be delivered in a suitable form, such as a fluid, a spray, an aerosol, an atomiser or a powder.

Upon delivery of the agent to the desired site of action, the device 100 may be moved to other sites if so desired, and eventually manually withdrawn from the airway.

It will also be appreciated that a similar device may be utilised which lacks the entry port 132, and which is used to first perturb a surface of the airway, then withdrawn from the airway, and the agent is delivered to the perturbed surface by delivery into the airway separately.

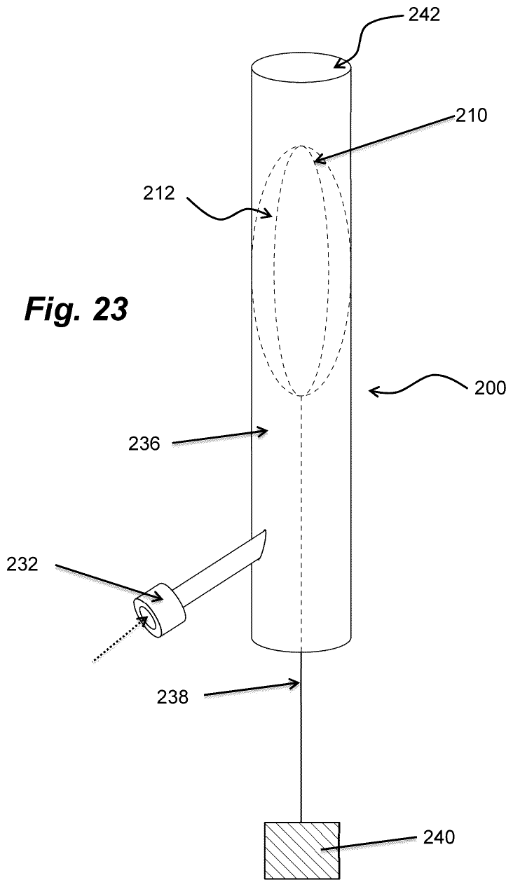

In another embodiment shown in FIG. 23, a device 200 for perturbing the airway utilises a single hollow tube 236 comprising an end component 210 for perturbing a surface of the airway. The device comprises a connector 238 attaching the end component 210 to a handle 240 allowing manual advancement and retraction of the end component 210 through the tube. In the embodiment shown, the end component 210 comprises a flexible and resilient cage 212, although other end components may used as described above.

The device may be manually advanced to a suitable site in the airway for perturbation, and the cage 212 advanced through tube 236, so that the cage 212 expands once it has emerged from the ending 242. In the expanded form, the cage 212 can be used to perturb the airway surface by abrasive and/or distensive action, particular by manually moving the cage 212 forwards and backwards in the airway. As indicated above, it will be appreciated that the device 200 may be adapted to utilise one of the other end components 210 described n Figure X.

The device 200 further utilises an entry port 232 to introduce the agent into the device 200 before, during and/or after perturbation of the airway has been conducted. In this case, the entry port 232 permits the agent to be introduced into the device 200 and to travel to the end of the device 242 along the inside of the tube 236, thereby delivering the agent to the area where the surface has been perturbed. Upon delivery of the agent, the cage 212 may be drawn into the tube 236 by compression, and the device 200 manually withdrawn from the airway, or moved to another desired site.

In a similar manner to that described above, a device similar to device 200 may be utilised which lacks the entry port 232, and the agent delivered to the perturbed surface by delivery into the airway.

A further embodiment of a device for perturbing a surface of an airway is shown in FIG. 23. In this embodiment, a device 300 for perturbing the airway utilises a bronchoscope 344 to allow visual placement of the device at a suitable location in the airway.

The device comprises expandible wire loops 346 to perturb a surface of the airway. The loops 346 and 348 may be held in a contracted position against the bronchoscope tube 350 when the bronchoscope is being positioned to a suitable part of the airway. The loops 346 may then be manually or electromechanically activated to move into an expanded position 350, where the loops 346 sit proud of the tube 348 and can be used to cause abrasive or distensive perturbation, particular as the bronchoscope is moved along the airway. It will be appreciated that for loop 348, the expanded loop is not visible being perpendicular to the plane of the page. Once perturbation has been accomplished, the loops 346, 348 may be manually or electromechanically activated to retract so as to assist with withdrawal of the device 300 from the airway.

After withdrawal of the device 300, the agent may then be delivered to the perturbed surface as described herein.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A non-surgical method of delivering an agent to the respiratory system of a subject, the method comprising in vivo perturbing of the airway surface in one or more parts of the respiratory system in the subject and exposing the perturbed surface of the airway to the agent, thereby delivering the agent to the respiratory system of the subject.

2. The method according to claim 1, wherein the respiratory system comprises one or more of the lungs, the bronchi, the bronchioles, the trachea, the pharynx, the nasal system, and the sinuses.

3. The method according to claim 1, wherein the perturbing of the airway surface comprises perturbing one or more of airway cells, airway mucous layer, airway surface liquid, epithelial cells, ciliated epithelial cells, goblet epithelial cells, basal epithelial cells, ionocytes, brush cells and intermediate cells.

4. (canceled)

5. The method according to claim 1, wherein the perturbing of the airway surface comprises disrupting the integrity of the tight junctions between the surface epithelial cells of the airway and/or partial or substantially complete removal of the surface epithelium.

6. The method according to claim 1, wherein the perturbing of the surface of the airway comprises one or more of mechanical perturbation, expansive perturbation, distensive perturbation, gaseous perturbation, fluidic perturbation, chemical perturbation, enzymatic perturbation, laser light perturbation, heat perturbation, bronchial thermoplasty, abrasive perturbation, expansive perturbation, distensive perturbation, ultrasonic perturbation, delivery of a gas or fluid to the respiratory system under pressure, perturbation using a fluidic jet or gaseous jet, bronchoalveolar lavage, and use of a device to apply a perturbative action to the airway surface.

7. (canceled)

8. The method according to claim 1, wherein_(i) the airway surface is perturbed and the agent is subsequently or simultaneously exposed to the perturbed surface: or (ii) the agent is exposed to the airway surface, and then the surface of the airway is subsequently perturbed.

9. (canceled)

10. The method according to claim 1, wherein the agent comprises a nucleic acid, a virus, a packaged recombinant virus, a viral vector, a non-viral vector, a nanoparticle, a gene-editing agent, a small molecule, a drug, a protein, a lipid, or a cell.

11. The method according to claim 10, wherein: (i) the nucleic acid comprises naked RNA, naked DNA, or a lipid encapsulated form of RNA or DNA; (ii) the virus or viral vector is selected from a lentivirus, an adenovirus, an adeno-associated virus, a helper-dependent adenovirus, a herpes virus, a retrovirus, an alphavirus, a flavivirus, a rhabdovirus or a measles virus; and (iii) the cell is a stem cell or a progenitor cell.

12. (canceled)

13. (canceled)

14. The method according to claim 1, wherein the subject is suffering from, or susceptible to, a disorder that would benefit from the delivery of an agent to the respiratory system.

15. The method according to claim 1, wherein the subject is suffering from, or susceptible to, a pulmonary disorder.

16. The method according to claim 15, wherein the pulmonary disorder is a genetic pulmonary disorder.

17. The method according to claim 1, wherein the agent comprises a nucleic acid for expression and the method produces detectable expression from the nucleic acid in the respiratory system for at least 7 days.

18. The method according to claim 1, wherein the method is used to improve delivery of an agent to a subject, for administration of an agent to a subject, for gene or cell delivery to the subject, to edit a gene in a subject, to deliver therapeutic cells to the subject, or to treat a subject suffering from, or susceptible to, a disorder.

19. (canceled)

20. (canceled)

21. (canceled)

22. (canceled)

23. A device for perturbing the airway surface in vivo in a subject, wherein the device in use produces an uncontrolled or controlled abrasive, expansive and/or distensive perturbation to the surface of the airway in the subject.

24. (canceled)

25. The device according to claim 23, wherein the device comprises a bronchoscope.

26. The device according to claim 23, wherein the device comprises an abrasive and/or an expandable component for perturbing the surface of the airway.

27. The device according to claim 23, wherein the device comprises a cage, a loop, or a brush configured for use in the respiratory system.

28. The device according to claim 23, wherein the device comprises a port to permit introduction of an agent in a vehicle into the device, and an outlet to permit delivery of the agent in the vehicle to the perturbed surface of the airway.

29. A device for delivering an agent to the respiratory system, the device comprising:
a component for perturbing a surface of the airway of the respiratory system; and
a port for introducing an agent into the device to deliver the agent to a perturbed surface; and
and an outlet to permit delivery of the agent in the vehicle to the perturbed surface of the airway.

30. (canceled)

31. A non-surgical method of delivering an agent in vivo to a subject, the method comprising using a device according to claim 29 to perturb the surface of the airway in the subject and exposing the perturbed surface of the airway to the agent, thereby delivering the agent to the subject.
